SEQUENCE LISTING (37 sequences)

SEQ ID NO: 1 - protein, 588 aa, Nicotiana tabacum
MDNSRTAFSDSNDISGSSSICCIGGGMTESFSPETSPAEITSLKRLSETLESIFDAASPEFDYFADAKLVIPGAGKEIPVHRCILSARSPFFKNLFCGKKEKNSSKVELKEVMKEYEVSYDAVVSVLAYLYSGKIRPSPKDVCVCVDNECSHVACRPAVAFLVEVLYISFTFQISELVDKFQRHLLDILGKAAADDVMMVLSVANICGKACERLLSSCIEIIVKSNVDIITLDKALPHDIVKQITDSRAELGLQGPESNGFPDKHVKRIHRALDSDDVELLQMLLREGHTTLDDAYALHYAVAYCDAKTTAELLDLALADVNHQNSRGYTVLHVAAMRKEPKIIVSLLTKGARPSDLTSDGRKALQIAKRLTRLVDFSKSPEEGKSASKDRLCIEILEQAERRDPLLGEASVSLAMAGDDLRMKLLYLENRVGLAKLLFPMEAKVAMDIAQVDGTSEFPLASISKKMVNAQRTTVDLNEAPFRIKEEHLNRLRALSRTVELGKRFFPRCSEVLNKIMDADDLSEIAYMGNDTAEERQLKKQRYMELQEILTKAFTEDKEEFDKTNNISSSCSSTSKGVDKPNKLPFRK

SEQ ID NO: 2 - protein, 593 aa, Arabidopsis thaliana
MDTTIDGFADSYEISSTSFVATDNTDSSIVYLAAEQVLTGPDVSALQLLSNSFESVFDSPDDFYSDAKLVLSDGREVSFHRCVLSARSSFFKSALAAAKKEKDSNNTAAVKLELKEIAKDYEVGFDSVVTVLAYVYSSRVRPPPKGVSECADENCCHVACRPAVDFMLEVLYLAFIFKIPELITLYQRHLLDVVDKVVIEDTLVILKLANICGKACMKLLDRCKEIIVKSNVDMVSLEKSLPEELVKEIIDRRKELGLEVPKVKKHVSNVHKALDSDDIELVKLLLKEDHTNLDDACALHFAVAYCNVKTATDLLKLDLADVNHRNPRGYTVLHVAAMRKEPQLILSLLEKGASASEATLEGRTALMIAKQATMAVECNNIPEQCKHSLKGRLCVEILEQEDKREQIPRDVPPSFAVAADELKMTLLDLENRVALAQRLFPTEAQAAMEIAEMKGTCEFIVTSLEPDRLTGTKRTSPGVKIAPFRILEEHQSRLKALSKTVELGKRFFPRCSAVLDQIMNCEDLTQLACGEDDTAEKRLQKKQRYMEIQETLKKAFSEDNLELGNSSLTDSTSSTSKSTGGKRSNRKLSHRRR

SEQ ID NO: 3 - protein, 597 aa, Arabidopsis thaliana
MMATTTARFSDSSYEFSNTSGNSFFAAESSIDYPSEFLTPPEVSALQLLSNCLESVFDSPENFYSDAKLVLAGGREVSFHRCILSARIPVFKSALATVKEQKSTTVKLEMKKIATDYEVGFDSVAAVLAFVYSGRVRPPPKGASDCVDDDCCHVACRPKVDFMVEVLYLAFVFQIPELVTMYERQFLKIVDKVVVEDILVIFKLDTLCGQTYKKLLDRCIEIIVKSDIELVSLEKSLPQHFVKQITGIRKALGLEPPELQIHVKNLYKALDSDDVELVKMLLLEGHTNLDMAYALHFAIAHCDVKTAYDLLELELADVNHRNPRGYTVLHVAAMRKEPKLIISLLMKGANVLDTLLDGRTALVIAKRLTKTDDYKTSMEDGTHSLKGGLCIEVLEHEQKLEYVLPREASLSLPVTPEELRMMLLYYENRVALARLLFPVESEIVQDIAKLDETCEFTASSLEPDHRIGEKRTSLDLNMAPFQIHEKHLSRLRAVCKTVELGKRYFRRCSLDHFMDTEDLNHLASVEEDTPEKRLQKKQRYVELQETLMKTFSEDKEEFGKPSTAKPTSAMRSNRKLSHRRLRVDKRDFLKRPCGNGD

SEQ ID NO: 4 - protein, 586 aa, Arabidopsis thaliana
MATLTEPSSSLSFTSSHFSYGSIGSNHFSSSSASNPEVVSLTKLSSNLEQLLSNSDCDYSDAEIIVDGVPVGVHRCILAARSKFFQDLFKKEKKISKTEKPKYQLREMLPYGAVAHEAFLYFLSYIYTGRLKPFPLEVSTCVDPVCSHDCCRPAIDFVVQLMYASSVLQVPELVSSFQRRLCNFVEKTLVENVLPILMVAFNCKLTQLLDQCIERVARSDLYRFCIEKEVPPEVAEKIKQLRLISPQDEETSPKISEKLLERIGKILKALDSDDVELVKLLLTESDITLDQANGLHYSVVYSDPKVVAEILALDMGDVNYRNSRGYTVLHFAAMRREPSIIISLIDKGANASEFTSDGRSAVNILRRLTNPKDYHTKTAKGRESSKARLCIDILEREIRKNPMVLDTPMCSISMPEDLQMRLLYLEKRVGLAQLFFPTEAKVAMDIGNVEGTSEFTGLSPPSSGLTGNLSQVDLNETPHMQTQRLLTRMVALMKTVETGRRFFPYGSEVLDKYMAEYIDDDILDDFHFEKGSTHERRLKRMRYRELKDDVQKAYSKDKESKIARSCLSASSSPSSSSIRDDLHNTT

SEQ ID NO: 5 - protein, 574 aa, Arabidopsis thaliana
MAATAIEPSSSISFTSSHLSNPSPVVTTYHSAANLEELSSNLEQLLTNPDCDYTDAEIIIEEEANPVSVHRCVLAARSKFFLDLFKKDKDSSEKKPKYQMKDLLPYGNVGREAFLHFLSYIYTGRLKPFPIEVSTCVDSVCAHDSCKPAIDFAVELMYASFVFQIPDLVSSFQRKLRNYVEKSLVENVLPILLVAFHCDLTQLLDQCIERVARSDLDRFCIEKELPLEVLEKIKQLRVKSVNIPEVEDKSIERTGKVLKALDSDDVELVKLLLTESDITLDQANGLHYAVAYSDPKVVTQVLDLDMADVNFRNSRGYTVLHIAAMRREPTIIIPLIQKGANASDFTFDGRSAVNICRRLTRPKDYHTKTSRKEPSKYRLCIDILEREIRRNPLVSGDTPTCSHSMPEDLQMRLLYLEKRVGLAQLFFPAEANVAMDVANVEGTSECTGLLTPPPSNDTTENLGKVDLNETPYVQTKRMLTRMKALMKTVETGRRYFPSCYEVLDKYMDQYMDEEIPDMSYPEKGTVKERRQKRMRYNELKNDVKKAYSKDKVARSCLSSSSPASSLREALENPT

SEQ ID NO: 6 - protein, 80 aa, Nicotiana tabacum
SASKDRLCIEILEQAERRDPLLGEASVSLAMAGDDLRMKLLYLENRVGLAKLLFPMEAKVAMDIAQVDGTSEFPLASISK

SEQ ID NO: 7 - protein, 80 aa, synthetic construct
SASKDRLCIEILEQAERRDPLLGEASVSLAMAGDDLRMKLLYLEKRVGLAKLLFPMEAKVAMDIAQVDGTSEFPLASISK

SEQ ID NO: 8 - protein, 79 aa, synthetic construct
SASKDRLCIEILEQEERRDPLLGEASVSLAMAGDDLRMKLLYLERVGLAKLLFPMEAKVAMDIAQVDGTSEFPLASISK

SEQ ID NO: 9 - protein, 80 aa, synthetic construct
SASKDRLCIEILEQEERRDPLLGEASVSLAMAGDDLRMKLLYLEKRVGLAKLLFPMEAKVAMDIAQVDGTSEFPLASISK

SEQ ID NO: 10 - protein, 61 aa, synthetic construct
PLLGEASVSLAMAGDDLRMKLLYLEKRVGLAKLLFPMEAKVAMDIAQVDGTSEFPLASISK

SEQ ID NO: 11 - protein, 52 aa, synthetic construct
LAMAGDDLRMKLLYLEKRVGLAKLLFPMEAKVAMDIAQVDGTSEFPLASISK

SEQ ID NO: 12 - protein, 73 aa, synthetic construct
SASKDRLCIEILEQEERRDPLLGEASVSLAMAGDDLRMKLLYLEKRVGLAKLLFPMEAKVAMDIAQVDGTSEF

SEQ ID NO: 13 - protein, 80 aa, synthetic construct; identical to SEQ ID NO: 9

SEQ ID NO: 14 - protein, 80 aa, synthetic construct; identical to SEQ ID NO: 9

SEQ ID NO: 15 - protein, 80 aa, synthetic construct; identical to SEQ ID NO: 9

SEQ ID NO: 16 - protein, 80 aa, synthetic construct
SASKDRLCIEILEQEERRDPLLGEASVSLAMAGDDLRMKLLYLERRVGLAKLLFPMEAKVAMDIAQVDGTSEFPLASISK

SEQ ID NO: 17 - protein, 80 aa, synthetic construct
SASKDRLCIEILEQEERRDPLLGEASVSLAMAGDDLRRKLLYLEKRVGLAKLLFPMEAKVAMDIAQVDGTSEFPLASISK

SEQ ID NO: 18 - protein, 80 aa, synthetic construct
SASKDRLCIEILEQEERRDPLLGEASVSLAMAGDDLRRKLLYLERRVGLAKLLFPMEAKVAMDIAQVDGTSEFPLASISK

SEQ ID NO: 19 - protein, 123 aa, Nicotiana tabacum
KMVNAQRTTVDLNEAPFRIKEEHLNRLRALSRTVELGKRFFPRCSEVLNKIMDADDLSEIAYMGNDTAEERQLKKQRYMELQEILTKAFTEDKEEFDKTNNISSSCSSTSKGVDKPNKLPFRK

SEQ ID NO: 20 - protein, 123 aa, synthetic construct
KMVNAQRTTVDLNEAPFRIKEEHLNRLRALSRTVELGKRFFPRCSEVLNKIMDAEDLSEIAYMGNDTAEERQLKKQRYMELQEILTKAFTEDKEEFDKTNNISSSCSSTSKGVDKPNKLPFRK

SEQ ID NO: 21 - protein, 123 aa, synthetic construct
KMVNAQRTTVDLNEAPFRIKEEMLNRLRALSRTVELGKRFFPRCSEVLNKIMDADDLSEIAYMGNDTAEERQLKKQRYMELQEILTKAFTEDKEEFDKTNNISSSCSSTSKGVDKPNKLPFRK

SEQ ID NO: 22 - protein, 123 aa, synthetic construct
KMVNAQRTTVDLNEAPERIKEEHLNRLRALSRTVELGKRFFPRCSEVLNKIMDADDLSEIAYMGNDTAEERQLKKQRYMELQEILTKAFTEDKEEFDKTNNISSSCSSTSKGVDKPNKLPFRK

SEQ ID NO: 23 - protein, 123 aa, synthetic construct
KMVNAQRTTVDLNEAPFRIKEEHLNRLRALERTVELGKRFFPRCSEVLNKIMDADDLSEIAYMGNDTAEERQLKKQRYMELQEILTKAFTEDKEEFDKTNNISSSCSSTSKGVDKPNKLPFRK

SEQ ID NO: 24 - protein, 123 aa, synthetic construct
KMVNAQRTTVDLNEAPFRIEEEHLNRLRALSRTVELGKRFFPRCSEVLNKIMDADDLSEIAYMGNDTAEERQLKKQRYMELQEILTKAFTEDKEEFDKTNNISSSCSSTSKGVDKPNKLPFRK

SEQ ID NO: 25 - protein, 123 aa, synthetic construct
KMVNAQRTTVDLNEAPERIEEEMLNRLRALERTVELGKRFFPRCSEVLNKIMDAEDLSEIAYMGNDTAEERQLKKQRYMELQEILTKAFTEDKEEFDKTNNISSSCSSTSKGVDKPNKLPFRK

SEQ ID NO: 26 - protein, 123 aa, synthetic construct; identical to SEQ ID NO: 25

SEQ ID NO: 27 - protein, 123 aa, synthetic construct; identical to SEQ ID NO: 25

SEQ ID NO: 28 - protein, 123 aa, synthetic construct; identical to SEQ ID NO: 25

SEQ ID NO: 29 - protein, 102 aa, synthetic construct
KMVNAQRTTVDLNEAPERIEEEMLNRLRALERTVELGKRFFPRCSEVLNKIMDAEDLSEIAYMGNDTAEERQLKKQRYMELQEILTKAFTEDKEEFDKTNNI

SEQ ID NO: 30 - protein, 109 aa, synthetic construct
KMVNAQRTTVDLNEAPERIEEEMLNRLRALERTVELGKRFFPRCSEVLNKIMDAEDLSEIAYMGNDTAEERQLKKQRYMELQEILTKAFTEDKEEFDKTNNISSSCSST

SEQ ID NO: 31 - protein, 109 aa, synthetic construct; identical to SEQ ID NO: 30

SEQ ID NO: 32 - protein, 109 aa, synthetic construct; identical to SEQ ID NO: 30

SEQ ID NO: 33 - protein, 203 aa, Nicotiana tabacum
SASKDRLCIEILEQAERRDPLLGEASVSLAMAGDDLRMKLLYLENRVGLAKLLFPMEAKVAMDIAQVDGTSEFPLASISKKMVNAQRTTVDLNEAPFRIKEEHLNRLRALSRTVELGKRFFPRCSEVLNKIMDADDLSEIAYMGNDTAEERQLKKQRYMELQEILTKAFTEDKEEFDKTNNISSSCSSTSKGVDKPNKLPFRK

SEQ ID NO: 34 - protein, 207 aa, Arabidopsis thaliana
HSLKGRLCVEILEQEDKREQIPRDVPPSFAVAADELKMTLLDLENRVALAQRLFPTEAQAAMEIAEMKGTCEFIVTSLEPDRLTGTKRTSPGVKIAPFRILEEHQSRLKALSKTVELGKRFFPRCSAVLDQIMNCEDLTQLACGEDDTAEKRLQKKQRYMEIQETLKKAFSEDNLELGNSSLTDSTSSTSKSTGGKRSNRKLSHRRR

SEQ ID NO: 35 - protein, 215 aa, Arabidopsis thaliana
HSLKGGLCIEVLEHEQKLEYVLPREASLSLPVTPEELRMMLLYYENRVALARLLFPVESEIVQDIAKLDETCEFTASSLEPDHRIGEKRTSLDLNMAPFQIHEKHLSRLRAVCKTVELGKRYFRRCSLDHFMDTEDLNHLASVEEDTPEKRLQKKQRYVELQETLMKTFSEDKEEFGKPSTAKPTSAMRSNRKLSHRRLRVDKRDFLKRPCGNGD

SEQ ID NO: 36 - protein, 204 aa, Arabidopsis thaliana
ESSKARLCIDILEREIRKNPMVLDTPMCSISMPEDLQMRLLYLEKRVGLAQLFFPTEAKVAMDIGNVEGTSEFTGLSPPSSGLTGNLSQVDLNETPHMQTQRLLTRMVALMKTVETGRRFFPYGSEVLDKYMAEYIDDDILDDFHFEKGSTHERRLKRMRYRELKDDVQKAYSKDKESKIARSCLSASSSPSSSSIRDDLHNTT

SEQ ID NO: 37 - protein, 202 aa, Arabidopsis thaliana
EPSKYRLCIDILEREIRRNPLVSGDTPTCSHSMPEDLQMRLLYLEKRVGLAQLFFPAEANVAMDVANVEGTSECTGLLTPPPSNDTTENLGKVDLNETPYVQTKRMLTRMKALMKTVETGRRYFPSCYEVLDKYMDQYMDEEIPDMSYPEKGTVKERRQKRMRYNELKNDVKKAYSKDKVARSCLSSSSPASSLREALENPT

(19) United States
(12) Patent Application Publication
(10) Pub. No.: US 2026/0048070 A1
(43) Pub. Date: Feb. 19, 2026

(54) SALICYLIC ACID AND ASPIRIN AS INDUCERS OF BIOMOLECULAR PROXIMITY FOR BIOMEDICAL APPLICATIONS

(71) Applicant: The Texas A&M University System, College Station, TX (US)

(72) Inventors: Yubin Zhou, Bellaire, TX (US); Yun Huang, Bellaire, TX (US); Tianlu Wang, Houston, TX (US)

(21) Appl. No.: 19/289,945

(22) Filed: Aug. 4, 2025

Related U.S. Application Data
(60) Provisional application No. 63/684,543, filed on Aug. 19, 2024.

(51) Int. Cl. A61K 31/60; A61K 40/11; A61K 40/31; A61K 40/42; C07K 14/705; C12N 5/0783

(52) U.S. Cl. CPC A61K 31/60; A61K 40/11; A61K 40/31; A61K 40/4211; C07K 14/705; C12N 5/0636; C12N 2501/30; C12N 2510/00

(57) ABSTRACT

The present disclosure provides compositions and methods for inducing protein-protein heterodimerization in the presence of salicylic acid or a synthetic derivative thereof, useful for modulating signal transduction, protein activation, and gene expression in response to chemical stimuli.

### CROSS-REFERENCE TO RELATED APPLICATIONS

This application claims the benefit of U.S. Provisional Application No. 63/684,543, filed Aug. 19, 2024, herein incorporated by reference in its entirety.

### STATEMENT REGARDING FEDERALLY SPONSORED RESEARCH OR DEVELOPMENT

This invention was made with government support under grant R01GM144986 awarded by the National Institutes of Health. The government has certain rights in the invention.

### INCORPORATION OF SEQUENCE LISTING

A sequence listing contained in the file named “TAMC086US_ST26.xml” which is 39,316 bytes (measured in MS-Windows®) and created on Jul. 31, 2025, is filed electronically herewith and incorporated by reference in its entirety.

### FIELD OF THE INVENTION

The present disclosure relates to the field of molecular biology, more specifically compositions and methods related to engineering protein-protein heterodimerization and to systems for inducing signal transduction, protein activation, and gene expression in response to chemical stimuli.

### BACKGROUND

Proximity, or the physical closeness of molecules, is a common regulatory mechanism in biology. Many posttranslational modifications, such as phosphorylation, methylation, and acetylation, promote the proximity of molecules, thereby playing crucial roles in cellular processes. Chemically induced proximity (CIP) allows for precise temporal control of transcription, signaling cascades, chromatin regulation, protein folding, localization, and degradation, as well as a host of other biologic processes. As such, CIP has potential therapeutic applications in a wide range of fields. However, translation of CIP methodologies into clinical settings still faces challenges due to the intrinsic limitations of the ligands involved, including structural complexity, high cost, partial reversibility, potential adverse effects, and biocompatibility issues. Therefore, a continuing need exists to develop CIP systems with enhanced translational potential, capable of overcoming these barriers to accelerate clinical integration with greater efficacy and safety.

### SUMMARY OF THE INVENTION

The present disclosure provides A method for inducing a protein-protein interaction, the method comprising obtaining a cell comprising a first and a second engineered salicylic acid receptor protein; and exposing the cell to the presence of salicylic acid or a synthetic derivative to activate a protein-protein interaction between the first and a second engineered salicylic acid receptor protein in the presence of said salicylic acid or synthetic derivative thereof. In some embodiments, the first engineered salicylic receptor protein comprises a polypeptide sequence having at least 85%, 90%, 95%, or 98% identity to a polypeptide selected from the group consisting of SEQ ID NOs: 6-18; and the second engineered salicylic receptor protein comprises a polypeptide sequence having at least 85%, 90%, 95%, or 98% identity to a polypeptide selected from the group consisting of SEQ ID NO:19-32. In other embodiments, the first or second engineered salicylic receptor protein comprises: a polypeptide sequence having an asparagine to lysine mutation corresponding to position 430 of SEQ ID NO: 1; a polypeptide sequence having an alanine to glutamate mutation corresponding to position 400 of SEQ ID NO: 1; a polypeptide sequence having an aspartate to glutamate mutation corresponding to position 520 of SEQ ID NO: 1; a polypeptide sequence having a histidine to methionine mutation corresponding to position 488 of SEQ ID NO: 1; a polypeptide sequence having a phenylalanine to glutamate mutation corresponding to position 482 of SEQ ID NO: 1; a polypeptide sequence having a serine to glutamate mutation corresponding to position 496 of SEQ ID NO: 1; a polypeptide sequence having a lysine to glutamate mutation corresponding to position 485 of SEQ ID NO: 1; a polypeptide sequence having an asparagine to arginine mutation corresponding to position 430 of SEQ ID NO: 1; a polypeptide sequence having an methionine to arginine mutation corresponding to position 423 of SEQ ID NO: 1; or a combination of any thereof. In further embodiments, the first engineered salicylic receptor protein comprises: a polypeptide sequence having an asparagine to lysine mutation corresponding to position 430 of SEQ ID NO: 1; a polypeptide sequence having an alanine to glutamate mutation corresponding to position 400 of SEQ ID NO: 1; a polypeptide sequence having an asparagine to arginine mutation corresponding to position 430 of SEQ ID NO: 1; a polypeptide sequence having an methionine to arginine mutation corresponding to position 423 of SEQ ID NO: 1; or a combination of any thereof. In certain embodiments, the second engineered salicylic receptor protein comprises: a polypeptide sequence having an aspartate to glutamate mutation corresponding to position 520 of SEQ ID NO: 1; a polypeptide sequence having a histidine to methionine mutation corresponding to position 488 of SEQ ID NO: 1; a polypeptide sequence having a phenylalanine to glutamate mutation corresponding to position 482 of SEQ ID NO: 1; a polypeptide sequence having a serine to glutamate mutation corresponding to position 496 of SEQ ID NO: 1; a polypeptide sequence having a lysine to glutamate mutation corresponding to position 485 of SEQ ID NO: 1; or a combination of any thereof. In other embodiments, the cell comprises a recombinant DNA construct encoding the first engineered salicylic acid receptor protein, the second engineered salicylic acid receptor protein, or the first and the second engineered salicylic acid receptor protein. In particular embodiments, the recombinant DNA construct is comprised within a vector; or the cell's genome. In certain embodiments, the recombinant DNA construct encodes the first engineered salicylic acid receptor protein and a second recombinant DNA construct encodes the second engineered salicylic acid receptor protein. In some embodiments, the first engineered salicylic acid receptor protein or the second engineered salicylic acid receptor protein is operably linked to a polypeptide sequence. In other embodiments, the protein-protein interaction between the first engineered salicylic receptor protein and the second engineered salicylic receptor protein modulates a cell signaling pathway or T cell activation. In specific embodiments, the cell signaling pathway comprises an ion-dependent, a MAPK-dependent, an ERK-dependent, or a phospholipase C gamma-dependent signaling pathway. In additional embodiments, the protein-protein interaction is fully reversible in the absence of said salicylic acid or the syn-thetic derivative thereof.

The present disclosure also provides a recombinant DNA construct encoding: a) a first engineered salicylic acid receptor protein comprising a polypeptide sequence having at least 85%, 90%, 95%, or 98% identity to a polypeptide selected from the group consisting of SEQ ID NOs: 6-18; b) a second engineered salicylic receptor protein comprising a polypeptide sequence having at least 85%, 90%, 95%, or 98% identity to a polypeptide selected from the group consisting of SEQ ID NOs: 19-32; or c) the first engineered salicylic acid receptor protein and the second engineered salicylic acid receptor protein recited in a) and b). In some embodiments, the recombinant DNA construct encoding the first engineered salicylic acid receptor protein or the second engineered salicylic acid receptor protein is operably linked in to a sequence encoding an enzyme, an antibody, a receptor, a transcription factor, a cytosolic protein, a membrane bound protein, or a proteasomal degradation component. In specific embodiments, the enzyme comprises a kinase; the antibody comprises a single chain variable fragment; the receptor comprises an engineered T cell receptor or co-stimulatory receptors; or the proteasomal degradation component comprises an E3 ligase, SKP1, DDB1, SPOP, or TRIM21. In particular embodiments, the kinase comprises a receptor tyrosine kinases selected from the group consisting of TrkA, FGFR, TrkB, VEGFR, and EGFR; the single chain variable fragment comprises a nanobody against mCherry, PD-1, PD-L1, Her-2, or CD19; or the engineered T cell receptor or co-stimulatory receptors comprises an effector domain comprising a 41BB, a CD28, a OX40, a ICOS, or a CD3ζ chain. In other embodiments the recombinant DNA construct encodes two or more copies of the first engineered salicylic acid receptor protein, the second engineered salicylic acid receptor protein, or two or more copies of both the first and second engineered salicylic acid receptor proteins. The present disclosure also provides cells comprising the DNA constructs described herein. In specific embodiments, the cell is a CAR T cell, a human cell, a bacterial cell, or a plant cell. In still further embodiments, the first engineered salicylic receptor protein and the second engineered salicylic receptor protein form a heterodimer in the presence of salicylic acid or a synthetic derivative thereof. In certain embodiments, the synthetic derivative is aspirin.

The present disclosure further provides A method for activating a CAR T cell in a patient, the method comprising: a) administering a CAR T cell to a patient, wherein the CAR T cell comprises a recombinant DNA construct encoding a first engineered salicylic acid receptor protein, a second engineered salicylic acid receptor protein, or a first and a second engineered salicylic acid receptor protein; and b) exposing the cell to the presence of salicylic acid or a synthetic derivative to activate a protein-protein interaction between the first and a second engineered salicylic acid receptor protein in the presence of said salicylic acid or synthetic derivative thereof; wherein activating the protein-protein interaction results in T cell activation. In some embodiments, the recombinant DNA construct encoding the first engineered salicylic acid receptor protein or the second engineered salicylic acid receptor protein is operably linked in to sequence encoding a T cell receptor-derived subunit, a costimulatory domain, or a tyrosine kinase domain, or an E3 ligase complex component. In further embodiments, the T cell receptor-derived subunit comprises a CD3ζ subunit; the costimulatory domain comprises a 4-1BB domain, a CD28 domain, an OX40, or a ZAP70; or the tyrosine kinase comprises the intracellular kinase domain of a tyrosine receptor kinase selected from the group consisting of TrkA, TrkB, EGFR, FGFR, VEGFR, and MET receptor tyrosine kinase. In particular embodiments, the patient does not exhibit significant weight loss; exhibits decreased release of CRS-associated cytokines; or a combination thereof; as compared to an appropriate control patient.

### BRIEF DESCRIPTION OF THE DRAWINGS

For a more complete understanding of the features and advantages of the present disclosure, reference is now made to the detailed description of the disclosure along with the accompanying figures. The drawings described herein are for illustration purposes only and are not intended to limit the scope of the present teachings in any way.

FIG. 1: Design, optimization and characterization of SAMBA. Panel a. Shows a cytosol-to-plasma membrane (PM) translocation assay used to screen split NPR protein pairs for designing a salicylic acid-mediated binary association (SAMBA) system. When salicylic acid (SA) promotes the reassembly of split NPR, it is expected to trigger the translocation of the mCherry-tagged N domain of the SA-binding region of NPR (mCh-NPR-N) toward the C domain of NPR tethered to the PM via a PM-targeting CAAX motif (NPR-C-CAXX). Panel b. Heatmap visualization representing the extent of SA-induced cytosol-to-PM translocation, quantified as the ratio of mCherry clearance in the cytosol before and after treatment with 1 mM SA, for the specified combinations. Assays were conducted in HeLa cells co-expressing both the N- and C-terminal fragments of NPR. Each combination was repeated eight times. Panel c. The predicted 3D structure of the SA-binding core region of NtNPR1 (aa 386-588) complexed with salicylic acid (SA; ball-and-stick), highlighting six α-helices (α1 to α6) and key residues mutated to create SAMBA. The model was generated based on the template from AtNPR4 (PDB entry: 6WPG) using I-TASSER. Panel d. Evolving SAMBA through three rounds of screening, involving rationalized engineering within the ligand-binding pocket and inter-helical interfaces, N/C-domain truncation, and subsequent randomized mutagenesis. Panel e. Dose response curves for the wild type (WT) split NtNPR1 protein and SAMBA. n=8 cells from three independent biological replicates (mean±SEM). Panel f. Visual illustration of the range of reported salicylate toxicity doses. The SAMBA working window was fine-tuned (indicated by the arrow) to be above the plasma SA concentration found in vegetarians (0.04 to 2.5 μM) to prevent background activation, while remaining well below the toxic range (>2 mM). Panel g. Quantification of cytosolic mCherry signal changes as exemplified in panel i. n=8 cells from three independent biological replicates (mean±SEM). Panel h. Quantification of the cytosolic mCherry-SAMBA-N clearance ratio following the addition of 100 μM SA (red), aspirin (blue), or other SA analogs (black). n=8 cells from three independent biological replicates (mean±SEM).

FIG. 2. SAMBA applied to reprogram ATP-fueled protein oscillation dynamics. Scale bar, 5 μm. Panel a. Shows Illustration of the minD/minE-based protein oscillator, driven by the reversible ATP/ADP-dependent association/dissociation of MinD ATPase with the mammalian cell endomembrane system. This dynamic process is tightly regulated by MinE as an ATPase-activating protein. Relocalization of cytosolic MinE to an organelle (such as the cytosolic side of ER) is induced by SA-mediated interaction between cytosolic MinE-SAMBA-N (MinE-N) and ER-anchored SAMBA-C (ER-C), thereby disrupting the MinDE circuit and reducing the oscillation frequency. Panel b. Cartoon depiction of key constructs used in the assay. Panel c. Real-time monitoring of mCh-MinD fluorescence signals at selected endomembrane areas following SA addition to HEK293T cells, as shown in panel c. Typical kymographs were shown above the graph. Panel d. Quantification of mCh-MinD oscillation frequency before and after SA treatment. n=8 cells from three independent biological replicates (mean±SEM). The P values were calculated using the two-sided unpaired Student's t-test. Panel e. Schematic illustrating the design of an SA-inducible, ON-switch MinDE circuit by engineering SAMBA into MinE. Upon SA addition, the split MinE is reassembled to restore its ATPase-activating function, subsequently triggering mCh-MinD oscillation (red). Panel f. Cartoon depiction of constructs used to generate SA-dependent MinDE oscillators. The N- and C-domains of SAMBA, connected by a P2A self-cleaving peptide, were inserted into two loop positions of MinE: between Q55 and I56 for MinE-55 and at G70/D71 for MinE-70. These insertion sites are indicated as magenta spheres in the 3D structure of MinE (PDB entry: 1EV0). Panels g and h. Time course showing SA-inducible mCh-MinD oscillation in HEK239T cells co-expressing either MinE-55 (Panel i) or minE-70 (Panel j). Typical kymographs were shown above the graphs. Panel i. Quantification of mCh-MinD oscillation frequency before and after SA treatment in HEK293T cells co-expressing MinE-55 or MinE-70. n=8 cells from three independent biological replicates (mean±SEM). The P values were calculated using the two-sided unpaired Student's t-test.

FIG. 3. SAMBA enables graded activation of Ca2+channels and NFAT. Panel a. Schematic depicting the graded activation of ORAI Ca2+channels in response to SA. SAMBA-C is fused individually to ER-resident modules with different oligomeric states, including the monomeric control (ER-C), dimeric GCN4 (C-GCN4, shown in panel b), trimeric foldon (C-Foldon), and the p53 tetramerization domain (C-TD). Upon SA addition, stromal interaction molecule 1 cytoplasmic domain (STIM1ct; aa 233-685) fused with SAMBA-N (abbreviated as N-ST1) docks toward the ER-bound components, thereby initiating oligomerization-induced conformational changes to activate ORAI channels to varying degrees. The resulting influx of extracellular Ca2+further activates the calmodulin (CaM)-dependent phosphatase, calcineurin (CaN), which dephosphorylates nuclear factor of activated-T cells (NFAT) and promotes NFAT nuclear entry and subsequent gene transcription. Panel b. Constructs used in the assay and summary of Ca2+/NFAT activation. SP, the ER-targeting signal peptide from STIM1; TM, the single transmembrane domain from STIM1; TD, tetramerization domain from p53. Panel c. Quantification of cytosolic calcium increase upon SA treatment in HeLa cells expressing the indicated constructs. n=36-60 cells from three independent biological replicates (mean±SEM). Panels d and e. I-V relationships of CRAC currents (ICRAC) recorded in HEK293T cells co-expressing N-ST1 with the indicated ER-resident constructs. Quantification of ICRAC was shown on the below bar graph. n=4 cells from 4 independent biological replicates (mean±SEM). Panel f. Quantification of the degree of nuclear accumulation of NFAT-GFP following the addition of 200 μM SA in HeLa cells expressing the indicated constructs. n=40-60 cells from three independent biological replicates (mean±SEM).

FIG. 4. SAMBA allows SA-tunable activation of TrkA signaling and gene expression. Panel a. Schematic of SA-induced TrkA activation via conditional recruitment of two copies of the intracellular kinase domain (TrkA-ICD) toward PM using the SAMBA system (SAMBA-N+2×SAMBA-C-CAAX; abbreviated as WT-N+2CX as shown in panel b). Calcium influx (Readout 1a), NFAT nuclear translocation (Readout 1b) and subsequent NFAT-dependent gene expression (EGFP or luciferase as reporters; Readouts 3a and 3b) indicate signaling through Y785 phosphorylation; whereas ERK-dependent nuclear export of a synthetic kinase activity relocation sensor (ERK-SKARS; Readout 2) reflects signaling via Y490 phosphorylation. Panel b. Constructs used in the assays depicted in panel a. The Y2F-N construct bearing Y490F/Y785F mutations was used as a negative control. Panel c. Quantification of cytosolic Ca2+changes following SA addition and withdrawal in HeLa cells co-expressing the indicated constructs. n=40-60 cells from three independent biological replicates (mean±SEM). Panel d. Quantification of nuclear accumulation of NFAT-GFP following SA addition to HeLa cells expressing the indicated constructs. n=40-60 cells from three independent biological replicates (mean±SEM). The P values were calculated using the two-sided unpaired Student's t-test. Panel e. Quantification of the nuclear clearance of ERK-SKARS signals in HeLa cells co-expressing the indicated constructs. n=40-60 cells from three independent biological replicates (mean±SEM). The P values were calculated using the two-sided unpaired Student's t-test. Panel f. Quantification of luminescence signals of HEK293T cells co-expressing the indicated constructs in the presence of escalating doses of SA. The domain architecture of the reporter construct was shown above the bar graph. n=3 independent biological replicates (mean±SEM). Panel g. Schematic depicting experimental procedures for in vivo evaluation of luciferase expression. HEK293T cells co-expressing WT-N, 2CX, and a luciferase reporter were subcutaneously injected into the right flanks of three groups of SCID mice, which received oral administration of PBS, salicylic acid (SA; 100 mg/kg), or aspirin (ASA; 100 mg/kg), respectively. The bioluminescence signals were collected after 16 h. Panel h. Quantification of bioluminescence signals from confocal images of EGFP reporter expression before and after SA treatment (HEK293T cells were co-transfected with the WT-N and 2CX constructs plus the EGFP reporter, the expression of which is under the control of three synthetic transcriptional response elements (RE) derived from serum response factor (SRE), NFAT, and the cAMP response element-binding (CRE) protein. The domain architecture of the reporter construct was shown above the images. Scale bar, 100 μm. n=5 independent mice (mean±SEM). The P values were calculated using the two-sided unpaired Student's t-test.

FIG. 5. Design Of Samba-Car T Cells For Inducible Tumor Killing. Panel a. Design of CARs that are dually gated by tumor-associated antigen (such as CD19) and SA. Engineered CAR T cells can only be switched on in the presence of SA or aspirin when engaged with cognate antigen-bearing tumor cells. Panel b. Constructs used in this study. Based on the conventional CAR or ZAP CAR, the reprogrammed CAR constructs were split into two parts (Part I and Part II). Part I constructs consist of the anti-CD19 scFv, a hinge and transmembrane segment, the 4-1BB costimulatory motif and SAMBA-N/C. To switch on the split CAR, Part II constructs (2N or 2C) contain a T cell receptor CD3ζ signaling chain, 4-1BB or the kinase and interdomain B domains derived from ZAP-70, and SAMBA-C or N fragment. For the defective constructs (2ND or 2CD), CD3ζ was removed from 2N/2C. For 2NP, the homodimeric DAP10 ectodomain was added to the N-terminal of 2N to retain Part II at the plasma membrane. Panel c. Quantification of the changes in cytosolic mCherry signal changes from 2N in response to repeated SA addition and withdrawal from the culture media. n=8 cells from three independent assays (mean±SEM). Panel d. NFAT-Luc reporter gene expression and IL-2 production were employed as measures to evaluate T cell activation following incubation with CD19+ Raji cells or CD19− K562 cells that either included or excluded SA. The level of activation is depicted by the intensified shading of the boxes. Panel e. Quantification of IL-2 production in Jurkat T cells with stable expression of the relevant constructs. Engineered T cells were co-cultured with tumor cells bearing the CD19 antigen (depicted as blue circles; hCD19+ Raji cells) or without the CD19 antigen (represented by open circles; hCD19− K562 cells), under conditions with (depicted as red squares) or without (depicted as open squares) 500 μM SA. Panel f. Dose-dependent IL2 production in Jurkat T cells coexpressing 1C and 2N in response to co-culture with Raji cells and various concentrations of SA. n=3 independent biological replicates (mean±SEM). Panel g. A schematic depiction of SAMBA-CAR T cell-mediated cytotoxicity against tumor cells. Panel h. Quantification of CAR-T mediated cytotoxicity against luciferase-expressing Raji lymphoma cells. WT or SAMBA CAR-T cells were co-cultured with Raji tumor cells, with the cell viability of Raji cells measured by bioluminescence signal changes. n=3 independent biological replicates (mean±SEM). Panel i. Experimental setup to evaluate the in vivo efficacy of engineered CAR-T cells. Panel j. Quantification of tumor weights on day 24. n=5 biologically independent mice (mean±SEM). The P values were calculated using the two-sided unpaired Student's t-test. Panel k. Measurement of tumor size from day 3 to day 24 every 3 or 4 days after tumor inoculation. n=5 biologically independent mice (mean±SEM; The tumour sizes at the indicated time points were measured using a digital caliper with the tumour volume calculated using the formula: length×width2×0.5). The P values were calculated using the two-sided unpaired Student's t-test.

FIG. 6. In vivo attenuation of cytokine release syndrome by SAMBA-CAR T Cells. Panel a. Experimental setup to establish the CRS mouse model. Raji cells (3 million) were intraperitoneally (i.p.) injected into SCID-beige mice. Following 3 weeks of tumor growth, WT CAR T cells or SAMBA-CAR T cells (30 million/group) were administered (i.p.) to induce acute cytokine storm. Subsequently, these mice received daily oral administration of SA. The body weight was monitored daily following CAR T cell injection. Blood/serum samples were collected on day 21 (pre-CAR implantation) and day 24 from anesthetized mice for ELISA assays. Panel b. Monitoring body weight changes following WT (blue) or SAMBA-CAR T cell (blue) injection from day 21 to day 24. n=3 biologically independent mice (mean±SEM). The P values were calculated using the two-sided unpaired Student's t-test. Panel c. Quantification of serum cytokine levels by ELISA. Cytokines (mIL-6, hIL-2, and hIFNγ) in the blood were detected 5 h before (Pre-CAR; black) or 24 h after injection of WT CAR T cells (blue) or SAMBA CAR-T cells (red) with SA treatment. n=3 biologically independent mice (mean±SEM). The P values were calculated using the two-sided unpaired Student's t-test.

FIG. 7. Primary sequence alignment of nonexpressor of pathogenesis-related genes 1-4 derived from Arabidopsis thaliana (AtNPR1-4) and Nicotiana tabacum NPR1 (NtNPR1). The split sites used in the current study were indicated by dashed red lines (Related to FIG. 1; Panel b). The level of conservation across these primary sequences was represented by the height of the bars.

FIG. 8 Truncated variants of NtNPR1. Panel a. Schematic illustration of NtNPR1 containing seven mutations (NtNRP1-7M) and six additional truncation variants (TR1-6) tested in the study. N and C denote the indicated N- and C-terminal parts of engineered NtNPR1. Panel b. Quantification of the cytosolic mCherry clearance ratio induced by 0.1 mM SA for the indicated truncated variants.

FIG. 9 I-V plot for ORAI1/STIM1-expressing HEK293T cells transfected with N-ST1 and the indicated ER-C variants in the absence of SA treatment (Related to FIG. 3; Panel d). n=4 from three independent biological replicates (mean±SEM).

FIG. 10 Dose response curve obtained from HEK293T cells (Related to FIG. 4; Panel f). Cells were co-transfected with WT-N, 2CX, and the luciferase reporter upon incubation with increasing concentrations of SA (0, 10, 100, 500, and 1000 PM). n=3 from three independent biological replicates (mean±SEM).

FIG. 11 Quantification of NFAT-dependent luciferase expression under the indicated conditions (Related to FIG. 5; Panel d). Measurement of NFAT-Luc reporter activity was performed in Jurkat T cells with stable expression of the indicated constructs. Engineered Jurkat cells were co-cultured with human CD19 (hCD19)-positive Raji cells (indicated by solid blue circle) or hCD19-negative K562 cells (open blue circle), in the absence (open red square) or presence (solid red square) of 500 μM salicylic acid (SA). n=3 from 3 independent biological replicates (mean±SEM).

FIG. 12 Dose-dependent NFAT-Luc reporter activity (Panel a) and IL2 production (Panel b) in Jurkat T cells co-expressing the indicated constructs (Related to FIG. 5; Panels d and e). n=3 from independent biological replicates (mean±SEM).

FIG. 13 Shows the universal genetic code chart showing all possible mRNA triplet codons (where T in the DNA molecule is replaced by U in the RNA molecule) and the amino acid encoded by each codon.

### BRIEF DESCRIPTION OF THE SEQUENCES

SEQ ID NO:1 is the polypeptide sequence of NtNPR-1.
SEQ ID NO:2 is the polypeptide sequence of AtNPR1.
SEQ ID NO:3 is the polypeptide sequence of AtNPR2.
SEQ ID NO:4 is the polypeptide sequence of AtNPR3.
SEQ ID NO:5 is the polypeptide sequence of AtNPR4.
SEQ ID NO:6 is the polypeptide sequence of the NtNPR1 wt N-terminal SAMBA protein.
SEQ ID NO:7 is the polypeptide sequence of the NtNPR1 N430K N-terminal SAMBA protein.
SEQ ID NO:8 is the polypeptide sequence of the NtNPR1 A400E N-terminal SAMBA protein.
SEQ ID NO:9 is the polypeptide sequence of the NtNPR1 7M N-terminal SAMBA protein.
SEQ ID NO:10 is the polypeptide sequence of the NtNPR1 7M-TR1 N-terminal SAMBA protein.
SEQ ID NO:11 is the polypeptide sequence of the NtNPR1 7M-TR2 N-terminal SAMBA protein.
SEQ ID NO:12 is the polypeptide sequence of the NtNPR1 7M-TR3 N-terminal SAMBA protein.
SEQ ID NO:13 is the polypeptide sequence of the NtNPR1 7M-TR4 N-terminal SAMBA protein.
SEQ ID NO:14 is the polypeptide sequence of the NtNPR1 7M-TR5 N-terminal SAMBA protein.
SEQ ID NO:15 is the polypeptide sequence of the NtNPR1 7M-TR6 N-terminal SAMBA protein.
SEQ ID NO:16 is the polypeptide sequence of the NtNPR1 7M-TR5-N430R N-terminal SAMBA protein.
SEQ ID NO:17 is the polypeptide sequence of the NtNPR1 7M-TR5-M423R N-terminal SAMBA protein.
SEQ ID NO:18 is the polypeptide sequence of the NtNPR1 7M-TR5-M423R-N430R N-terminal SAMBA protein.
SEQ ID NO:19 is the polypeptide sequence of the NtNPR1 wt C-terminal SAMBA protein.
SEQ ID NO:20 is the polypeptide sequence of the NtNPR1 D520E C-terminal SAMBA protein.
SEQ ID NO:21 is the polypeptide sequence of the NtNPR1 H488M C-terminal SAMBA protein.
SEQ ID NO:22 is the polypeptide sequence of the NtNPR1 F482E C-terminal SAMBA protein.
SEQ ID NO:23 is the polypeptide sequence of the NtNPR1 S496E C-terminal SAMBA protein.
SEQ ID NO:24 is the polypeptide sequence of the NtNPR1 K485E C-terminal SAMBA protein.
SEQ ID NO:25 is the polypeptide sequence of the NtNPR1 7M C-terminal SAMBA protein.
SEQ ID NO:26 is the polypeptide sequence of the NtNPR1 7M-TR1 C-terminal SAMBA protein.
SEQ ID NO:27 is the polypeptide sequence of the NtNPR1 7M-TR2 C-terminal SAMBA protein.
SEQ ID NO:28 is the polypeptide sequence of the NtNPR1 7M-TR3 C-terminal SAMBA protein.
SEQ ID NO:29 is the polypeptide sequence of the NtNPR1 7M-TR4 C-terminal SAMBA protein.
SEQ ID NO:30 is the polypeptide sequence of the NtNPR1 7M-TR5 C-terminal SAMBA protein.
SEQ ID NO:31 is the polypeptide sequence of the NtNPR1 7M-TR6 C-terminal SAMBA protein.
SEQ ID NO:32 is the polypeptide sequence of the NtNPR1 7M-TR5-M423R-N430R C-terminal SAMBA protein.
SEQ ID NO:33 is a polypeptide sequence comprising amino acids 386-588 of NtNPR1.
SEQ ID NO:34 is a polypeptide sequence comprising amino acids 387-593 of AtNPR1.
SEQ ID NO:35 is a polypeptide sequence comprising amino acids 383-597 of AtNPR2.
SEQ ID NO:36 is a polypeptide sequence comprising amino acids 383-586 of AtNPR3.
SEQ ID NO:37 is a polypeptide sequence comprising amino acids 373-574 of AtNPR4.

### DETAILED DESCRIPTION

Following the introduction of the binary FRB-FKBP dimerization system more than two decades ago, several additional CIP systems have been described. Despite this progress, the translation of CIP methodologies into clinical settings still faces challenges due to the intrinsic limitations of these systems and the respective ligands involved (e.g. structural complexity, high cost, partial reversibility, potential adverse effects, and biocompatibility issues). Therefore, a pressing need exists to develop CIP systems with enhanced translational potential, capable of overcoming these barriers to accelerate clinical integration with greater efficacy and safety.

The present disclosure overcomes the limitations of the prior art by providing significantly improved methods for inducing protein-protein interactions as well as related compositions. The Salicylic Acid-Mediated Binary Association (SAMBA) system described herein permits rapid and reversible heterodimerization of engineered split components in a chemically inducible manner. In particular, the present disclosure enables rapid protein-protein heterodimerization in response to SA or synthetic derivatives thereof. These methods and compositions represent a highly efficient chemically-induced proximity (CIP) system, having broad applicability to control various biological processes both in vitro and in vivo.

The present invention was developed through a combination of rational engineering and random mutagenesis targeting a plant SA receptor, the NONEXPRESSOR OF PATHOGENESIS-RELATED 1 (NPR1); and disclosure provided herein supports the system's broad adaptability. For example, SAMBA has been operably linked to various signaling molecules, including ATPases, ion channel complexes, and membrane receptors as described herein. These integrations yield tunable control over protein activity, cell signaling, and gene expression, both in vitro and in vivo, with high temporal precision. Further, provided herein are programmable chimeric antigen receptor (CAR) T cells that respond to SA or aspirin, termed SAMBA-CAR T cells. These engineered cells empower precise targeting and suppression of tumor growth while significantly mitigating the risk of cytokine release syndrome (CRS)—a common side effect associated with CAR T cell therapy.

The present disclosure therefore expands the CIP toolkit by providing an economical, flexible, and easily adaptable chemogenetic platform, catering to a broad spectrum of applications that span from fundamental research to therapeutic innovations. Notably, some embodiments of the present invention comprise the minimal SAMBA variant, comprising only 189 amino acids, which ranks among the most compact CIP systems and is compatible with various viral packaging platforms. Its small size also makes it easier for potential CRISPR-mediated integration into endogenous genomic loci. These strengths, along with the ability to utilize one of the smallest molecular switches for inducing protein-protein interaction (SA) as the chemical switch to trigger reversible protein-protein heterodimerization, make SAMBA an incredibly versatile system for achieving precise temporal control over cellular processes without causing unintended systemic toxicity. As such, provided herein are methods for inducing a protein-protein interaction comprising obtaining a cell comprising a first and a second engineered salicylic acid receptor protein; exposing the cell to the presence of salicylic acid or a synthetic derivative thereof; and activating a protein-protein interaction between the first engineered salicylic receptor protein and the second engineered salicylic receptor protein in the presence of said salicylic acid or synthetic derivative thereof.

The ability to operably link a first and/or a second engineered salicylic acid receptor protein to a further polypeptide sequence (also referred to as an amino acid sequence) as described herein enables the system to reprogram protein oscillations, modulate cell signaling, control kinase activation, and tune gene expression. Remarkably, SAMBA exhibits a 150-fold increase in potency compared to the native SA receptor, thereby affording precise temporal control of protein-protein interactions with activation and deactivation half-lives on the order of seconds to minutes.

### I. Engineered Proteins and Recombinant DNA Molecules

Provided herein are novel, engineered proteins and the recombinant DNA molecules that encode them. As used herein, the term “engineered” refers to a non-natural DNA, protein, cell, or organism that would not normally be found in nature and was created by human intervention. An “engineered protein,” or “engineered salicylic acid receptor protein,” refers to a protein whose amino acid sequence was conceived of and created in the laboratory using one or more of the techniques of biotechnology, protein design, or protein engineering, such as molecular biology, protein biochemistry, bacterial transformation, plant transformation, site-directed mutagenesis, directed evolution using random mutagenesis, genome editing, gene editing, gene cloning, DNA ligation, DNA synthesis, protein synthesis, and DNA shuffling. For example, an engineered protein may have one or more deletions, insertions, or substitutions relative to the coding sequence of the wild-type protein and each deletion, insertion, or substitution may consist of one or more amino acids. Genetic engineering can be used to create a DNA molecule encoding an engineered protein, such as a first or a second engineered salicylic acid receptor protein and comprises at least a first amino acid substitution relative to a wild-type salicylic acid receptor protein protein as described herein.

Examples of engineered proteins provided herein are salicylic acid receptor proteins having salicylic acid binding activity (referred herein as “engineered salicylic acid receptor protein”) comprising at least 70% sequence identity to an amino acid sequence selected from the group consisting of SEQ ID NOs:1-5 and 33-37, wherein the protein comprises at least one amino acid substitution as compared to SEQ ID NO:1, and wherein the protein comprises: a glutamate (E) at the position corresponding to position 400 of SEQ ID NO:1; an arginine (R) at the position corresponding to position 423 of SEQ ID NO:1; a lysine (K) or arginine (R) at the position corresponding to position 430 of SEQ ID NO:1; a glutamate (E) at the position corresponding to position 482 of SEQ ID NO:1; a glutamate (E) at the position corresponding to position 485 of SEQ ID NO:1; a methionine (M) at the position corresponding to position 488 of SEQ ID NO:1; a glutamate (E) at the position corresponding to position 496 of SEQ ID NO:1; or a glutamate (E) at the position corresponding to position 520 of SEQ ID NO:1. In specific embodiments, an engineered protein provided herein comprises one, two, three, four, five, six, seven, eight, nine, ten, or more of any combination of such substitutions.

Engineered proteins are proteins that dimerize in the presence of a chemical stimuli. As used herein, engineered proteins have the ability to interact with other engineered proteins in the presence of salicylic acid or synthetic derivative thereof.

As used herein, “wild-type” means naturally-occurring. As used herein, a “wild-type DNA molecule”, “wild-type polypeptide”, or a “wild-type protein” is a naturally-occurring DNA molecule, polypeptide, or protein, that is, a DNA molecule, polypeptide, or protein pre-existing in nature. A wild-type version of a polypeptide, protein, or DNA molecule may be useful for comparison with an engineered protein or gene. An example of a wild-type protein useful for comparison with the engineered proteins provided by the present disclosure is the NONEXPRESSOR OF PATHOGENESIS-RELATED 1 (NPR1) from Nicotiana tabacum. An example of a wild-type DNA molecule useful for comparison with the recombinant DNA molecules provided by the present disclosure is the NPR1 gene from Nicotiana tabacum.

As used herein, “control” means an experimental control designed for comparison purposes. For example, a control cell is a cell of the same type as the experimental cell (that is, the cell to be tested; prokaryotic or eukaryotic) but does not contain the genetic insert, recombinant DNA molecule, DNA construct, or variant protein or gene of the experimental cell. Examples of control cells useful for comparison include wild type CAR T cells. As used herein, the term “recombinant” refers to a non-naturally occurring DNA, protein, cell, or organism that is the result of genetic engineering and was created by human intervention. A “recombinant DNA molecule” is a DNA molecule comprising a DNA sequence that does not naturally occur and as such is the result of human intervention, such as a DNA molecule comprising at least two DNA molecules heterologous to each other. An example of a recombinant DNA molecule is a DNA molecule provided herein encoding a first engineered salicylic acid receptor protein or a second engineered salicylic acid receptor protein operably linked to a heterologous promoter. A “recombinant protein” is a protein comprising an amino acid sequence that does not naturally occur and as such is the result of human intervention, such as an engineered protein. A recombinant cell, or organism is a cell or organism comprising transgenic or heterologous DNA or protein, for example a transgenic cell, or organism comprising a DNA construct or engineered protein of the present disclosure.

As used herein, the term “DNA” or “DNA molecule” refers to a double-stranded DNA molecule of genomic or synthetic origin (that is, a polymer of deoxyribonucleotide bases or a polynucleotide molecule) read from the 5′ (upstream) end to the 3′ (downstream) end. As used herein, the term “DNA sequence” refers to the nucleotide sequence of a DNA molecule. The nomenclature used herein corresponds to that of by Title 37 of the United States Code of Federal Regulations § 1.822, and set forth in the tables in WIPO Standard ST.25 (1998), Appendix 2, Tables 1 and 3.

The present disclosure provides a nucleic acid molecule encoding a first engineered salicylic acid receptor protein comprising a polypeptide sequence (also referred to as an amino acid sequence) having at least 85%, 86%, 87%, 88%, 89%, 90%, 91%, 92%, 93%, 94%, 95%, 96%, 97%, 98%, 99%, or 100%, identity to a polypeptide selected from the group consisting of SEQ ID NO:6-18; a second engineered salicylic receptor protein comprising a polypeptide sequence (also referred to as an amino acid sequence) having at least 85%, 86%, 87%, 88%, 89%, 90%, 91%, 92%, 93%, 94%, 95%, 96%, 97%, 98%, 99%, or 100% identity to a polypeptide selected from the group consisting of SEQ ID NO:19-32; or a first engineered salicylic acid receptor protein and a second engineered salicylic acid receptor protein as described. In some embodiments, said nucleic acid molecule can be described as comprising a sequence encoding a polypeptide sequence having an asparagine to lysine mutation corresponding to position 430 of SEQ ID NO:1; a polypeptide sequence having an alanine to glutamate mutation corresponding to position 400 of SEQ ID NO:1; a polypeptide sequence having an aspartate to lysine mutation corresponding to position 430 of SEQ ID NO:1; a polypeptide sequence having an asparagine to glutamate mutation corresponding to position 520 of SEQ ID NO:1; a polypeptide sequence having a histidine to methionine mutation corresponding to position 488 of SEQ ID NO:1; a polypeptide sequence having a phenylalanine to glutamate mutation corresponding to position 482 of SEQ ID NO:1; a polypeptide sequence having a serine to glutamate mutation corresponding to position 496 of SEQ ID NO:1; a polypeptide sequence having a lysine to glutamate mutation corresponding to position 485 of SEQ ID NO:1; a polypeptide sequence having an asparagine to arginine mutation corresponding to position 430 of SEQ ID NO:1; a polypeptide sequence having an methionine to arginine mutation corresponding to position 423 of SEQ ID NO:1; or a combination of any thereof, and otherwise comprising a sequence encoding a polypeptide sequence having at least 70%, 75%, 80%, 85%, 86%, 87%, 88%, 89%, 90%, 91%, 92%, 93%, 94%, 95%, 96%, 97%, 98%, 99%, or 100%, identity to SEQ ID NO:1.

As used herein, the term “protein-coding DNA molecule” refers to a DNA molecule comprising a DNA sequence that encodes a protein. As used herein, the term “protein” refers to a chain of amino acids linked by peptide (amide) bonds and includes both polypeptide chains that are folded or arranged in a biologically functional way and polypeptide chains that are not. As used herein, a “protein-coding sequence” means a DNA sequence that encodes a protein. As used herein, a “sequence” means a sequential arrangement of nucleotides or amino acids. A “DNA sequence” may refer to a sequence of nucleotides or to the DNA molecule comprising of a sequence of nucleotides; a “protein sequence” may refer to a sequence of amino acids or to the protein comprising a sequence of amino acids. The boundaries of a protein-coding sequence are usually determined by a translation start codon at the 5′-terminus and a translation stop codon at the 3′-terminus.

Engineered proteins may be produced by changing or modifying a wild-type protein sequence to produce a new protein with modified characteristic(s) or a novel combination of useful protein characteristics, such as altered substrate specificity, substrate selectivity, ability to interact with other components in the cell such as partner proteins or membranes, and protein stability, among others. Modifications may be made at specific amino acid positions in a protein and may be made by substituting an alternate amino acid for the typical amino acid found at that same position in nature (that is, in the wild-type protein). Amino acid modifications may be made as a single amino acid substitution in the protein sequence or in combination with one or more other modifications, such as one or more other amino acid substitution(s), deletions, or additions. In some embodiments, an engineered protein has altered protein characteristics, such as those that result in increased sensitivity to one or more chemical stimuli as compared to the wild-type protein or ability to interact with one or more additional engineered proteins in a cell expressing the engineered protein(s). In other embodiments, the present disclosure therefore provides an engineered protein such as a engineered salicylic receptor protein, and the recombinant DNA molecule encoding it, having one or more amino acid substitution(s) selected from the group consisting of A400E, M423R, N430R, N430K, F482E, K485E, H488M, S496E, D520E, and all combinations thereof, wherein the position of the amino acid substitution(s) is relative to the amino acid position set forth in SEQ ID NO:1. In specific embodiments, an engineered protein provided herein comprises one, two, three, four, five, six, seven, eight, nine, ten, or more of any combination of such substitutions, wherein the modification is made at a position relative to a position comparable in function to that in the amino acid sequence provided as SEQ ID NO:1.

As used herein, the term “isolated” refers to at least partially separating a molecule from other molecules typically associated with it in its natural state. As used herein, the term “isolated” refers to a DNA molecule that is separated from the nucleic acids that normally flank the DNA molecule in its natural state. For example, a DNA molecule encoding a protein that is naturally present in a bacterium would be an isolated DNA molecule if it was not within the DNA of the bacterium from which the DNA molecule encoding the protein is naturally found. Thus, a DNA molecule fused to or operably linked to one or more other DNA molecule(s) with which it would not be associated in nature, for example as the result of recombinant DNA or genetic transformation techniques, is considered isolated herein. Such molecules are considered isolated even when integrated into the chromosome of a host cell or present in a nucleic acid solution with other DNA molecules.

Any number of methods well known to those skilled in the art can be used to isolate and manipulate a DNA molecule, or fragment thereof, as disclosed herein. For example, polymerase chain reaction (PCR) technology can be used to amplify a particular starting DNA molecule or to produce variants of the original molecule. DNA molecules, or fragment thereof, can also be obtained by other techniques, such as by directly synthesizing the fragment by chemical means, as is commonly practiced by using an automated oligonucleotide synthesizer.

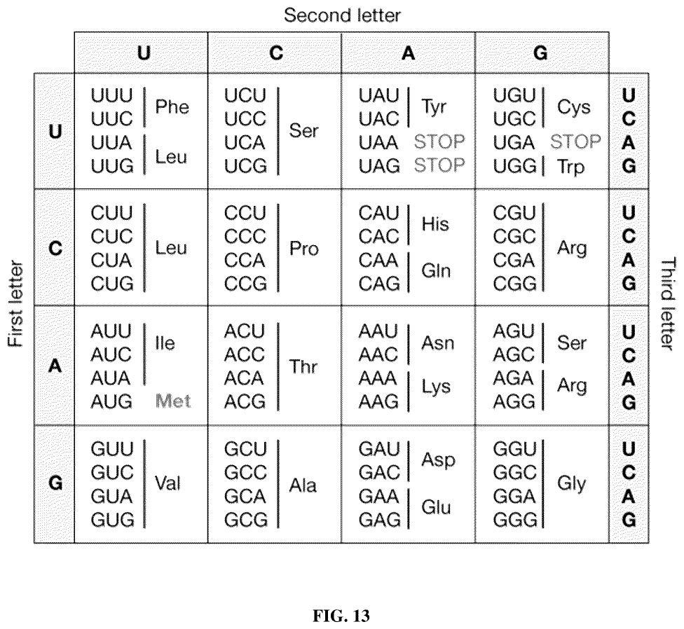

Because of the degeneracy of the genetic code, a variety of different DNA sequences can encode proteins, such as the engineered proteins disclosed herein. For example, FIG. 13 provides the universal genetic code chart showing all possible mRNA triplet codons (where T in the DNA molecule is replaced by U in the RNA molecule) and the amino acid encoded by each codon. DNA sequences encoding engineered salicylic acid receptor proteins described herein can be produced by introducing mutations into the DNA sequence encoding a wild-type salicylic acid receptor protein using methods known in the art and the information provided in FIG. 13. It is well within the capability of one of skill in the art to create alternative DNA sequences encoding the same, or essentially the same, altered or engineered proteins as described herein. These variant or alternative DNA sequences are within the scope of the embodiments described herein. As used herein, references to “essentially the same” sequence refers to sequences which encode amino acid substitutions, deletions, additions, or insertions that do not materially alter the functional activity of the protein encoded by the DNA molecule of the embodiments described herein. Allelic variants of the nucleotide sequences encoding a wild-type or engineered protein are also encompassed within the scope of the embodiments described herein. Substitution of amino acids other than those specifically exemplified or naturally present in a wild-type or engineered salicylic acid receptor protein are also contemplated within the scope of the embodiments described herein, so long as the engineered salicylic acid receptor protein having the substitution still retains substantially the same functional activity described herein.

Recombinant DNA molecules provided herein may be synthesized and modified by methods known in the art, either completely or in part, where it is desirable to provide sequences useful for DNA manipulation (such as restriction enzyme recognition sites or recombination-based cloning sites) or sequences useful for DNA construct design (such as spacer or linker sequences).

The present disclosure includes recombinant DNA molecules and engineered proteins having at least 50% sequence identity, at least 60% sequence identity, at least 70% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, and at least 99% sequence identity to any of the recombinant DNA molecule or amino acid sequences provided herein, and having protein-protein interaction activity in the presence of salicylic acid or synthetic derivative thereof (such as heterodimerization activity). As used herein, the term “percent sequence identity” or “% sequence identity” refers to the percentage of identical nucleotides or amino acids in a linear polynucleotide or amino acid sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, CA), MEGAlign (DNAStar Inc., 1228 S. Park St., Madison, WI 53715), and MUSCLE (version 3.6) (RC Edgar, “MUSCLE: multiple sequence alignment with high accuracy and high throughput” Nucleic Acids Research 32(5):1792-7 (2004)) for instance with default parameters. An “identity fraction” for aligned segments of a test sequence and a reference sequence is the number of identical components that are shared by the two aligned sequences divided by the total number of components in the portion of the reference sequence segment being aligned, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence.

### II. Expression Constructs

As used herein, a “DNA construct” is a recombinant DNA molecule comprising two or more heterologous DNA sequences. DNA constructs are useful for expression and may be comprised in vectors and plasmids. DNA constructs may be used in vectors for transformation (that is, the introduction of heterologous DNA into a host cell) to produce recombinant bacteria or transgenic cells (and as such may also be contained in the mitochondrial DNA or genomic DNA of a cell). As used herein, a “vector” means any recombinant DNA molecule that may be used for prokaryotic or eukaryotic transformation. DNA molecules provided herein can, for example, be inserted into a vector as part of a DNA construct having the DNA molecule operably linked to a heterologous gene expression element that functions in a cell, such as a mammalian cell, to affect expression of the engineered protein encoded by the DNA molecule. Methods for making and using DNA constructs and vectors are well known in the art and described in detail in, for example, handbooks and laboratory manuals including Michael R. Green and Joseph Sambrook, “Molecular Cloning: A Laboratory Manual” (Fourth Edition) ISBN:978-1-936113-42-2, Cold Spring Harbor Laboratory Press, NY (2012). The components for a DNA construct, or a vector comprising a DNA construct, include one or more gene expression elements operably linked to a transcribable nucleic acid sequence, such as the following: a promoter for the expression of an operably linked DNA, an operably linked protein-coding DNA molecule, and an operably linked 3′ untranslated region (UTR). Gene expression elements that are useful include, but are not limited to, one or more of the following type of elements: promoter, 5′ UTR, enhancer, leader, cis-acting element, intron, transit sequence, 3′ UTR, and one or more selectable marker transgenes.

As used herein, the term “heterologous” refers to the relationship between two or more things not normally associated in nature, for instance that are derived from different sources or not normally found in nature together in any other manner. For example, a DNA molecule or protein may be heterologous with respect to another DNA molecule, protein, cell, or organism if not normally found in nature together or in the same context. In certain embodiments, a first DNA molecule is heterologous to a second DNA molecule if the two DNA molecules are not normally found in nature together in the same context. For instance, a protein-coding recombinant DNA molecule is heterologous with respect to an operably linked promoter if such a combination is not normally found in nature. Similarly, a protein is heterologous with respect to a second operably linked protein, if such combination is not normally found in nature. In another embodiment, a recombinant DNA molecule encoding a first or a second engineered salicylic acid receptor protein is heterologous with respect to an operably linked promoter that is functional in a cell if such combination is not normally found in nature. A recombinant DNA molecule also may be heterologous with respect to a cell or organism into which it is inserted when it would not naturally occur in that cell or organism.

A “heterologous protein” is a protein present in a cell, tissue, or organism in which it does not naturally occur or operably linked to a protein with which it is not naturally linked. An example of a heterologous protein is a first or a second engineered salicylic acid receptor protein comprising at least a first amino acid substitution described herein that is expressed in any cell, tissue, or organism. Another example is a first or a second engineered salicylic acid receptor protein operably linked to a second protein, such as an enzyme, an antibody, a receptor, a transcription factor, a cytosolic protein, or a membrane bound protein, with which it is not naturally linked, or a protein introduced into a cell in which it does not naturally occur using the techniques of genetic engineering.

The first or the second engineered salicylic acid receptor protein can be operably linked to any polypeptide of interest, including but not limited to those described herein. In certain embodiments, a first or a second engineered salicylic acid receptor protein operably linked to a an enzyme, an antibody, a receptor, a transcription factor, a cytosolic protein, a membrane bound protein, or a proteasomal degradation component. For example, a recombinant DNA construct encoding the first engineered salicylic acid receptor protein or the second engineered salicylic acid receptor protein can be operably linked in to a sequence encoding an enzyme, an antibody, a receptor, a transcription factor, a cytosolic protein, a membrane bound protein, or a proteasomal degradation component.

As used herein, “operably linked” means two or more DNA molecules or two or more proteins linked in manner so that one may affect the function of the other. Operably linked DNA molecules or operably linked proteins may be part of a single contiguous molecule and may or may not be adjacent. For example, a promoter is operably linked with a protein-coding DNA molecule in a DNA construct where the two DNA molecules are so arranged that the promoter may affect the expression of the transgene.

The DNA constructs of the present disclosure may include a promoter operably linked to a protein-coding DNA molecule provided herein, whereby the promoter drives expression of the engineered protein. Promoters useful in practicing the contemplated embodiments include those that function in a cell for expression of an operably linked DNA molecule, such as a bacterial or eukaryotic promoter. Eukaryotic promoters are varied and well known in the art and include, for instance, those that are inducible, viral, synthetic, constitutive, temporally regulated, spatially regulated, or spatio-temporally regulated.

In some embodiments, a DNA construct provided herein includes a DNA sequence encoding a transit sequence that is operably linked to a heterologous DNA sequence encoding a first or a second engineered salicylic receptor protein, whereby the transit sequence facilitates localizing the protein molecule within the cell. Transit sequences are known in the art as signal sequences, targeting peptides, targeting sequences, localization sequences, and transit peptides. By facilitating protein localization within the cell, the transit sequence may increase the accumulation of recombinant protein, or protect the protein from proteolytic degradation, and thereby reduce levels of injury in the cell or organism.

As used herein, “transgene expression”, “expressing a transgene”, “protein expression”, and “expressing a protein” mean the production of a protein through the process of transcribing a DNA molecule into messenger RNA (mRNA) and translating the mRNA into polypeptide chains, which are ultimately folded into proteins. A protein-coding DNA molecule may be operably linked to a heterologous promoter in a DNA construct for use in expressing the protein in a cell transformed with the recombinant DNA molecule.

### III. Applications of the SAMBA System

Salicylic acid distinguishes itself among other existing inducers of CIP systems due to its cost-effectiveness, high biocompatibility, superior reversibility, smaller structure (MW of only 138 Da; among the smallest ligand), and predictable behaviors in human, positioning it as a promising candidate for future clinical applications.

The SAMBA engineered proteins described herein (i.e. the first and second engineered salicylic receptor proteins described herein) exhibit significantly improved SA binding affinity and specificity. These enhancements facilitate precise temporal control over protein interactions, with activation and deactivation half-lives on the order of seconds to minutes (57.4±3.1 s and 79.8±0.9 s for activation and deactivation half-lives), without causing unintended systemic toxicity. SAMBA demonstrates a rapid responsiveness to salicylic acid that is over 150-fold greater than that of the wild-type NtNPR1, enabling its use at concentrations (EC50=66.7 μM) significantly below the toxic doses (>2.2 mM). This moderate affinity is designed on purpose to avoid SAMBA being preactivated in mammals with high vegetable intake (serum maximal SA concentration reaching up to 2-10 μM in vegetarians). In brief, SAMBA permits rapid and reversible heterodimerization of two engineered split components in a strictly SA-dependent manner. For example, activating a protein-protein interaction between a first engineered salicylic receptor protein and a second engineered salicylic receptor protein in the presence of salicylic acid or synthetic derivative thereof.

As a versatile CIP system, SAMBA can be utilized to control a wide range of proteins (e.g. ATPase, ion channel, membrane receptors, and signaling molecules) to modulate the protein oscillation systems, gate ion channels on a graded scale, and induce expression of exogeneous genes in a salicylic acid-inducible manner, as well as tunable activation of therapeutic CAR T cells. In fact, the exemplary embodiments described herein show how the SAMBA system may be engineered into various signaling molecules, including ATPases, ion channel complexes, and membrane receptors. These integrations afford tunable control over protein activity, cell signaling, and gene expression, with high temporal precision.

### EXAMPLES

The following examples are included to demonstrate preferred embodiments of the present disclosure. It should be appreciated by those of skill in the art that the techniques disclosed in the examples, which follow represent techniques discovered by the inventors to function well in the practice of the present disclosure, and thus can be considered to constitute preferred modes for its practice. However, those of skill in the art should, in light of the present disclosure, appreciate that many changes can be made in the specific embodiments, which are disclosed and still obtain a like or similar result without departing from the spirit and scope of the present disclosure.

### Example 1: Materials and Methods

The following example describes the materials and methods used to develop and characterize the Salicylic Acid (SA)-Mediated Binary Association system (SAMBA).

### Chemical Reagents

Salicylic acid, sodium salicylate, aspirin, benzoic acid, 3-methylsalicylic acid, 3-aminosalicylic acid, 4-aminosalicylic acid, 5-aminosalicylic acid, 3,4-diaminobenzoic acid, 2,6-dihydroxybenzoic acid, 2,3-dihydroxybenzoic acid, 4-(dimethylamino) benzoic acid, and 4-(hydroxymethyl)benzoic acid were purchased from Sigma Aldrich (St. Louis, MO, USA).

### Molecular Cloning and Plasmids Construction

Plasmids construction was performed using a standard restriction enzyme digestion and ligation method. KOD Hot Start DNA polymerase was purchased from EMD Millipore (Burlington, MA, USA) and used for most PCR amplifications. Oligonucleotides were synthesized by Sigma Aldrich (St. Louis, MO, USA). The T4 DNA ligase kit and NEBuilder HiFi DNA Assembly Master Mix were purchased from New England BioLabs (Ipswich, MA, USA). gBlocks were purchased from GENEWIZ (South Plainfield, NJ, USA). QuikChange Multi Site-Directed Mutagenesis Kit and Random Mutagenesis Kit were obtained from Agilent Technologies (Santa Clara, CA, USA).

To construct plasmids utilized for NPR protein screening, gBlocks of 5 different NPR proteins were first individually inserted between mCherry and a CAAX motif in the pTriEx vector. Next, a P2A sequence was inserted between the specified split sites in loops regions of NPR proteins. The rest 20 plasmids encoding various NPR hybrid proteins were generated via HiFi DNA Assembly based on these five parental constructs. Plasmids for mutagenesis screening were generated by using the QuikChange Site-Directed Mutagenesis Kit and GeneMorph II Random Mutagenesis Kit. mCherry-MinD was constructed by inserting a gBlock of MinD into the pmCherry-C1 vector. MinE-EGFP-SAMBA-N was created through the stepwise insertion of the MinE gBlock and the amplified SAMBA-N fragment into the pEGFP-N1 vector. SP-TM-SAMBA-C was produced by amplifying the ER signal peptide (SP) and transmembrane domain (TM) of STIM1, followed by insertion into the pcDNA vector with an amplified SAMBA-C fragment. MinE-55/70-SAMBA-EGFP were made by inserting the SAMBA-N fragment, P2A sequence, and the SAMBA-C fragment into the corresponding split sites of MINE-EGFP. To construct mCherry-SAMBA-N-STIM1ct, amplified SAMBA-N fragment and STIM1 fragment (233-685) were inserted into the pmCherry-C1 vector. SP-TM-SAMBA-C-GCN4/Foldon/TD were made by fusing the corresponding genes to the C-terminus of SP-TM-SAMBA-C. mCherry-TrkA-ICD-SAMBA-N were constructed by inserting TrkA-ICD between mCherry and SAMBA-N in the pTriEx vector. 2×SAMBA-C-CAAX was produced by fusing two SAMBA-C fragments with a CAAX motif with subsequent insertion into the pTriEx vector. EGFP-ERK-SKARS was generated by inserting the gBlock fragment of MEK2 (residues 1-40) in front of two NLS sequence motifs within the pEGFP-C1 vector. The conventional CAR construct was made by inserting the anti-CD19 scFv, the hinge region, the transmembrane domain, the 4-1BB costimulatory motif, the human CD3ζ intracellular chain, and EYFP into the pWPXL lentiviral vector. ZAP-CAR was created by replacing the human CD3ζ intracellular chain with the kinase and interdomain B of ZAP-70. Part I constructs only contained the anti-CD19 scFv, the hinge region, the transmembrane segment, the 4-1BB costimulatory motif, and SAMBA-N/C compared to whole CAR constructs. Part II constructs (2N or 2C) contained a T cell receptor CD3ζ signaling chain, along with 4-1BB or the kinase domain and interdomain B of ZAP-70 besides SAMBA-C/N. For the defective constructs (2ND or 2CD), 4-1BB-CD3ζ was removed from constructs 2N or 2C.

### Cell Culture and Transfection

The HeLa and HEK293T cell lines were purchased from American Type Culture Collection (ATCC). Cells were cultured at 37° C. with 5% CO2 in Dulbecco's Modified Eagle medium (DMEM; Sigma-Aldrich; St. Louis, MO, USA) supplemented with 10% fetal bovine serum (FBS) and 1% penicillin/streptomycin cocktail. For fluorescence imaging experiments, cells were seeded in 35-mm glass-bottom dishes (Cellvis, Mountain View, CA, USA). When cells reaching about 50-70% confluency on day 2, transient transfection was performed using the Lipofectamine 3000 reagent (Life Technologies; Carlsbad, CA, USA) by following the manufacturer's instructions. 6 h post-transfection, cells were replenished with normal DMEM. On Day 3-4, transfected cells were mounted on a Nikon confocal microscope stage for imaging.

An NFAT-Luc reporter Jurkat cell line (jktl-nfat, InvivoGen) was used to examine NFAT-dependent gene transcription. Human cancer cell lines (K562 myelogenous leukemia cells (CCL-243), and Raji cell lymphoblasts (CCL-86)) were purchased from ATCC and cultured in Roswell Park Memorial Institute (RPMI 1640) medium with L-glutamine supplemented with 10% FBS, 100 U/ml penicillin and 100 g/ml streptomycin (Gibco).

### Live-Cell Imaging and Image Analysis

Fluorescence imaging was performed on a Nikon Ti2 Inverted microscope equipped with a Yokogawa W-1 dual spinning disk scanhead, Micro-Scanner for photo-stimulation and stage top incubator, along with a live-cell culture cage to maintain the temperature at 37° C. with 5% CO2. Confocal imaging was carried out using either a 60×oil or 40×oil lens. The half maximal effective concentration (EC50) values of SAMBA or WT-NtNPR1 were determined by incubating HeLa cells with DMEM media containing various concentrations of sodium salicylate for 2 min. To calculate the changes in cytosolic mCherry signal clearance (in the form of F0/F), the “Intensity Line Profile” function in the Nikon Elements software was employed. Titration curves were fitted using a dose-response curve function ([Agonist] vs. response—Variable slope (four parameters)) in the Prism 8 software. Eight cells were selected for each titration curve, and all experiments were independently repeated three times.

To monitor the MinDE circuit in HEK293T cells, cells were transfected with either mCherry-MinD, MinE-EGFP-SAMBA-N, and SP-TM-SAMBA-C, or mCherry-MinD along with MinE-55/70-SAMBA-EGFP. 488 nm and 561 nm laser sources were utilized for exciting EGFP and mCherry, respectively. During the imaging process, a final concentration of 500 μM SA was introduced to the transfected HEK293T cells. Time-lapse imaging was performed at 4-second intervals over a duration of up to 30 minutes.

To measure Ca2+influx in HeLa cells co-expressing the green calcium indicator GCaMP6s, SP-TM-SAMBA-C-GCN4/Foldon/TD, and mCherry-STIM1ct-SAMBA-N, 488-nm and 561-nm laser sources were utilized to excite EGFP and mCherry respectively after addition of 200 μM SA, with an 8-second interval between excitation events. The acquired images were subsequently analyzed using the NIS-Elements AR microscope imaging software (Nikon, NIS-element AR version 4.0). A selection of 40-60 cells was made to define regions of interest (ROI) for the analysis of GCaMP6s fluorescence intensity. These experiments were repeated three times. Similar procedures were employed to monitor Ca2+influx in HeLa cells transfected with mCherry-TrkA-ICD-SAMBA-N, 2×SAMBA-C-CAAX, and GCaMP6s.

To monitor the nuclear translocation of NFAT-GFP in response to SAMBA-controlled graded Ca2+influx, HeLa cells were transfected with NFAT1(1-460)-GFP, SP-TM-SAMBA-C-GCN4/Foldon/TD, and mCherry-STIM1ct-SAMBA-N. Imaging was conducted 24 h after transfection, and a total of 30 minutes of time-lapse imaging was captured at 15-second intervals. Increase of the nuclear NFAT (1-460)-GFP signal (presented as F/F0) were used to assess the efficiency of NFAT activation. A minimum of 40 cells were analyzed for each condition in three independent experiments. Similar procedures were employed to monitor NFAT activation in HeLa cells transfected with mCherry-TrkA-ICD-SAMBA-N, 2×SAMBA-C-CAAX, and NFAT (1-460)-GFP.

To monitor the activity changes of ERK in live HeLa cells for SAMAB-controlled TrkA signaling activation, HeLa cells were transfected with mCherry-TrkA-ICD-SAMBA-N, 2×SAMBA-C-CAAX and EGFP-ERK-SKARS. Imaging was conducted 24 h after transfection, and a total of 30 minutes of time-lapse imaging was captured at 15-second intervals. Clearance of the nuclear EGFP-ERK-SKARS signal (presented as F0/F) was used to assess the efficiency of ERK activation. A minimum of 40 cells were analyzed for each condition in three independent experiments.

CRAC Current Measurements with Patch-Clamp

Patch-clamp experiments were performed at 21-25° C. using the standard whole-cell recording configuration by using HEK EPC9 USB double patch amplifier controlled by the Patchmaster software (HEKA Elektronik) as previously described52. Only cells with high input resistance (>20) were selected for recording. Membrane potentials were corrected for a liquid junction potential of 10 mV. The holding potential was set to 0 mV, and currents were monitored by voltage ramps of 50 ms, spanning a range of −100 to +100 mV, applied at 2 s intervals over a period of 100-400 s. Currents were filtered at 2.9 kHz and digitized at a rate of 20 kHz. Currents obtained before the activation of CRAC channels were assigned as background/leak currents and subtracted from the subsequent recorded currents. The current densities accurately measured by correcting the leak currents collected in Ca2+Ringer's solution with 50 μM LaCl3. All patch clamp data analysis and curve fitting were done with Igor Pro 5.03 (WaveMetrics) and Prism (Version 8.0.0; GraphPad) softwares. The standard extracellular Ringer's solution contained 120 mM NaCl, 2 mM MgCl2 10 mM TEACL, 10 mM HEPES, 10 mM CaCl2), and 10 mM D-glucose (pH adjusted to 7.4 using NaOH). The standard intracellular solution contained 120 mM cesium-glutamate, 8 mM MgCl2, 10 mM BAPTA, and 10 mM HEPES (pH adjusted to 7.2 using CsOH).

### Ex Vivo Luciferase Reporter Activity Measurements

Following the transfection of HEK293T cells with mCherry-TrkA-ICD-WT-SAMBA-N, 2×SAMBA-C-CAAX, and the luciferase reporter, the cells were exposed to various concentrations of SA for a 16-h incubation period. Subsequently, luciferase activity was measured using the Bright-Glo Luciferase Assay System (Promega) and a Cytation 5 luminescence microplate reader (BioTek). The resulting data were analyzed and graphed using the Prism software (version 8.0.0, GraphPad).

### In Vivo Quantification of Luciferase Reporter Activity

Animal experiments in this study were conducted in compliance with the protocols approved by the Institutional Animal Care and Use Committee (IACUC) at the Institute of Biosciences and Technology, School of Medicine, Texas A&M University. The IACUC adheres to the National Institute of Health Guide for the Care and Use of Laboratory Animals, which is grounded in the United States Government Principles for the Utilization and Care of Vertebrate Animals Used in Testing, Research, and Training. The utilization of human cancer cell lines in this research was carried out in accordance with institutional guidelines governing human cell research and the approved protocol established by the Texas A&M University Institute of Biosciences and Technology.

In the xenograft mouse models, SCID-beige mice aged 4 to 8 weeks were subcutaneously injected into the right flank with 1×106HEK293T cells. These cells were transiently transfected with mCherry-TrkA-ICD-WT-SAMBA-N, 2×SAMBA-C-CAAX, and a luciferase reporter, at 16 h before the experiment. Subsequently, the mice were orally administered PBS, or 100 mg/kg SA or ASA for 16 h prior to being anesthetized. The mice were then subcutaneously injected with 150 mg of D-luciferin per kg body weight and subjected to in vivo imaging using the IVIS Lumina Series III system (Perkin Elmer). The resulting data were analyzed using the Living Image 4.5.2 software (Perkin Elmer).

### Quantification of NFAT-Luc Reporter Activity in Jurkat Cells

NFAT-luc reporter Jurkat T cells (jktl-nfat, InvivoGen) expressing conventional CARs or split CARs based on SAMBA (105 cells per well) were co-cultured with cognate CD19+ Raji cells or non-cognate CD19− K562 cells at the ratio of 1:3 in 96-well flat-bottom microplates (E17073EF, Greiner Bio-one). Cells were incubated at 37° C. in a humidified atmosphere under 5% CO2 with or without addition of SA. Luciferase activity was assayed by adding the coelenterazine substrate to a final concentration 50 μM and utilizing a Cytation 5 luminescence microplate reader (BioTek). Data plots were generated using the Prism software (version 8.0.0, GraphPad).

### ELISA Measurements of Cytokine Production

Jurkat T cells expressing conventional CARs or split CARs based on SAMBA (105 cells per well) were co-cultured with cognate CD19+ Raji cells or non-cognate CD19− K562 cells at the ratio of 1:3 in 96-well flat-bottom microplates. Plates with cells were incubated at 37° C. in a humidified atmosphere under 5% CO2 with or without addition of SA. The cell supernatants were collected and analyzed using the BD OptEIA Human IL-2 ELISA Set (431816, Biolegend) according to the manufacturer's instructions. The concentrations of samples were calculated from the standard curve, and the data were plotted using the Prism software (GraphPad).

### Isolation and Culture of Primary Human T Cells

Blood samples from de-identified individuals were obtained from healthy donors at the Gulf Coast Regional Blood Center in Houston, Texas, USA. The utilization of human blood adhered to the ethical standards outlined in the institutional guidelines for human cell research by the Institutional Review Board (IRB) of Texas A&M University. Peripheral blood mononuclear cells (PBMCs) were isolated by density gradient centrifugation using Ficoll-Paque Plus medium (Cytiva, #17544202). The buffy layer was collected and mixed with ACK lysing buffer (Gibco, A1049201). CD3+ T cells were subsequently enriched using CD3 magnetic microbeads (Miltenyi Biotec, 130-097-043) by following the manufacturer's instructions. For cell culture, CTS OpTmizer medium (Gibco, A1048501) supplied with 2 mM L-glutamine (Gibco, A2916801) and 200 IU/ml human IL-2 (Pepro Tech, 200-02) was employed. Dynabeads human T-activator CD3/CD38 (Gibco, 11131D) were added to the T cell culture medium at a bead-to-cell ratio of 1:1. T cells were cultured at a density ranging from 0.5 to 2×106 cells/ml.

### Lentivirus Packaging and Transduction

HEK293T cells (ATCC, CRL-3216) were co-transfected with lentivirus packaging and envelope plasmids (psPAX2 and pMD2.G) alongside the CAR/split CAR-encoding lentiviral vector (pWPXL) using the iMFectin DNA transfection reagent (GeneDEPOT, I7100-101). The virus-containing media were harvested 48 and 72 h post-transfection and filtered using 0.45 m syringe filters to remove cell debris. Lenti-X concentrator (Takara, 631232) was added to the viral supernatant at a ratio of 1:3 and incubated overnight at 4° C., followed by centrifugation at 1500 g for 1 h. The viral pellet was resuspended in T cell culture medium and stored at −80° C. until use. For transduction of Jurkat T cells, virus and 4-6 g/ml polybrene (EMD Millipore, TR-1003-G) were added to 12-well plates, then centrifuged at 2000 g for 2 h at 32° C. on two consecutive days. To transduce human CD3+ T cells, the concentrated virus was added to non-treated 24-well plates coated with the RetroNectin reagent (Takara, T100) and subjected to centrifugation at 2000 g for 2 h at 32° C. After virus removal, T cells were added to the plates and incubated at 37° C.

### Quantification of NFAT-Luciferase Reporter Activity in Jurkat Cells

Jurkat-Lucia NFAT Cells (InvivoGen, jktl-nfat), stably expressing either the conventional anti-CD19 CAR or split CAR, were co-cultured with CD19+ Raji cells or CD19− K562 cells at an effector-to-target (E:T) ratio of 1:3, with or without SA treatment, in 96-well flat-bottom microplates (Greiner Bio-one, E17073EF). The cell mixtures were then incubated at 37° C. for 18 hours. To assess luciferase activity, coelenterazine substrate (Promega, 52001) was added to cells with a final concentration of 50 μM, and the signal was recorded using the Cytation 5 luminescence microplate reader (BioTek). Data analysis and visualization were performed using the Prism software (Version 8.0.0, GraphPad).

### Cytotoxicity Assay

Human CD3+ T cells, transduced with either WT CAR or SAMBA-CAR variants, were co-cultured with luciferase-expressing tumor cells (Raji or K562 cells) at an E:T ratio of 10:1 for 18 h. The culture medium was supplemented with 50 μM SA or aspirin, or with DMSO serving as the control vehicle. Luciferase activity was quantified by adding D-luciferin (Goldbio, LUCK-100) at a final concentration of 150 g/ml. The bioluminescent signal was recorded using a Cytation 5 luminescence microplate reader (BioTek).

### Xenograft Mouse Model of Lymphoma

SCID mice (6 to 8 weeks; either female or male), purchased from Charles River Laboratories were subcutaneously (s.c.) injected with 3×105CD19+ Raji cells along with Matrigel matrix (Corning, CLS354234) into the dorsal flank. After 10 days, CAR-T or split CAR-T cells (2×106) were intravenously infused into tumor-bearing mice. SA dissolved in PBS or aspirin dissolved in mixture of PEG400 and PBS buffer was orally administrated to mice daily. Tumor burden was continuously monitored using a caliper, and tumor volume was calculated using the formula: length×width2×0.5. On day 24, mice were euthanized for tumor isolation and subsequent phenotypic analyses.

### Cytokine Release Assay

Serum samples were obtained from mouse blood and analyzed using ELISA kits for human IL-2 (BioLegend, 431816), human IFN-7 (BioLegend, 430116), and mouse IL-6 (BioLegend, 431304) according to the manufacturer's instructions. The concentrations of samples were calculated from the standard curve, and the data were plotted with the Prism software (GraphPad).

### Statistical Analysis

Quantitative data are presented as mean±SEM as indicated. The number of samples analyzed (n) for each experiment is detailed in the corresponding figure legends. Statistical analyses were performed using Prism (version 8.0.0; GraphPad, San Diego, CA, USA).

### Example 2: Design and Optimization of SAMBA

Through a combination of rational engineering and random mutagenesis, this example describes the development of the Salicylic Acid-Mediated Binary Association (SAMBA) system, based on a plant SA receptor, the NONEXPRESSOR OF PATHOGENESIS-RELATED 1 (NPR1). As described herein, SAMBA permits rapid and reversible heterodimerization of two engineered split components in a strictly SA-dependent manner. The broad adaptability of SAMBA is further described in Examples 3-7. These exemplary applications enable tunable control over protein activity, cell signaling, and gene expression, both in vitro and in vivo, with high temporal precision.

The crystal structure of the SA-binding core (SBC) domain from the N-terminal domain of Arabidopsis NPR4 (AtNPR4) reveals five closely packed α-helices with SA positioned at the tapered end of the four-helix bundle. In view of the extensive packing between helices observed in this structure, engineering a split NPR protein into an SA-mediated binary association (SAMBA) system was contemplated, which would utilize SA as a molecular switch to facilitate protein-protein heterodimerization.

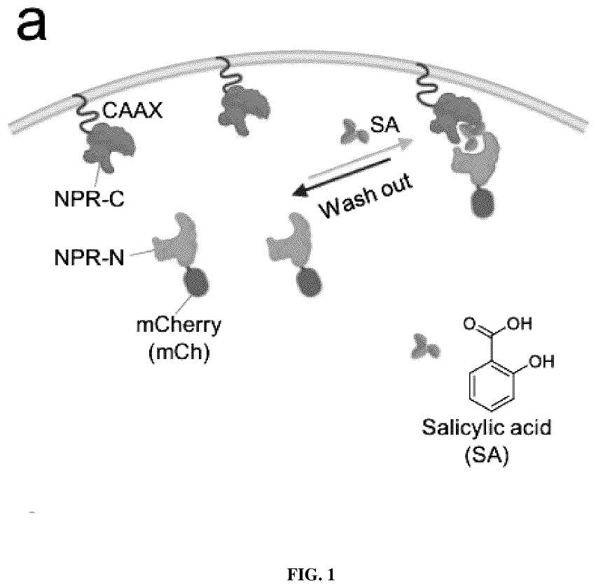

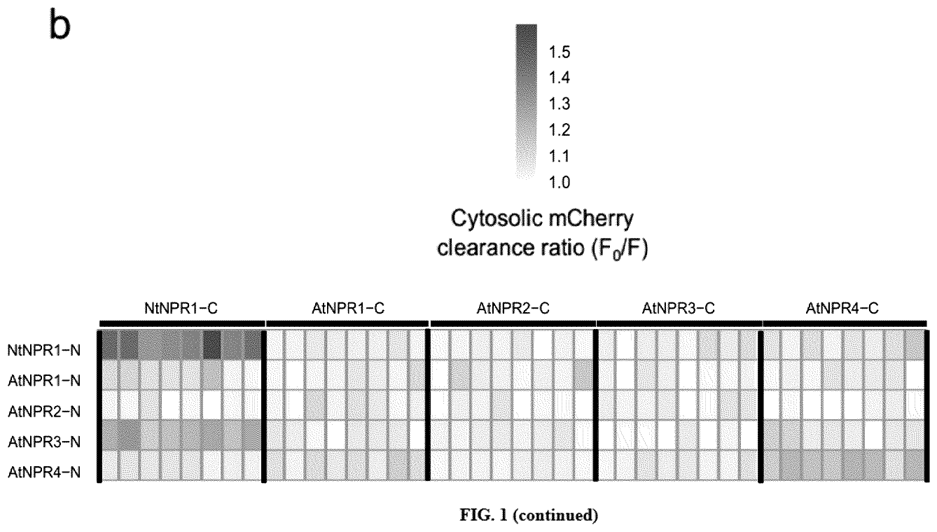

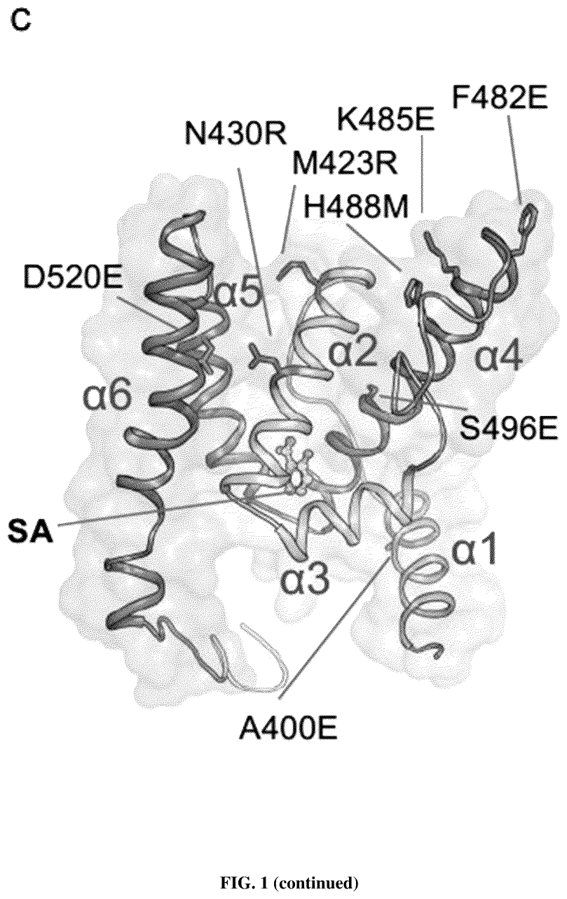

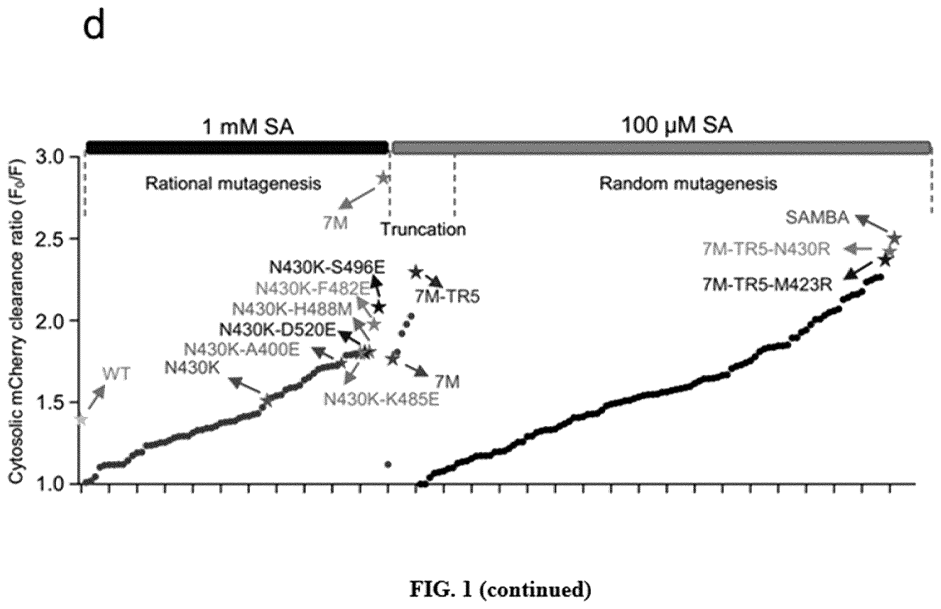

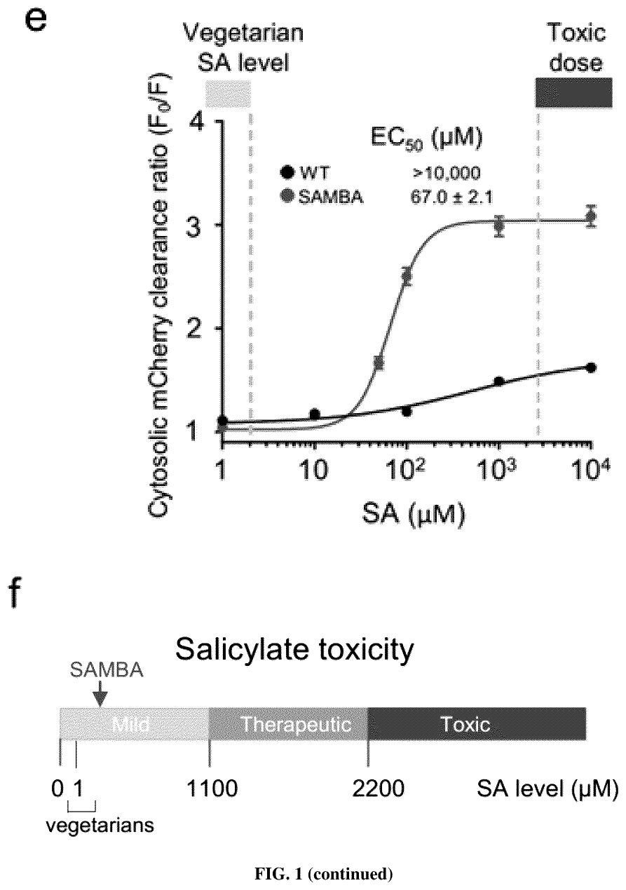

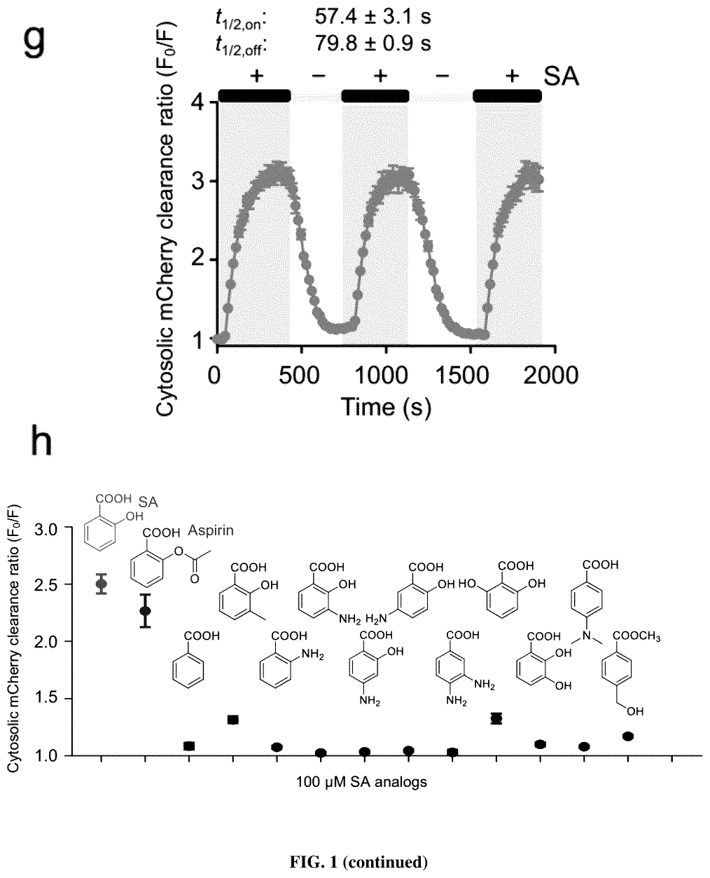

To evaluate the effectiveness of SA-induced proximity, a cytosol-to-plasma membrane (PM) translocation assay was established, where the C-terminal half of the SBC region of NPR proteins (NPR-C) is anchored to the PM through a CAAX motif, while the N-terminal domain (NPR-N) is tagged with mCherry for visual monitoring of its subcellular location (FIG. 1; Panel a). By measuring the clearance of cytosolic mCherry intensity due to PM translocation, SA-inducible heteromerization of proteins could be quantitatively assessed in real time.

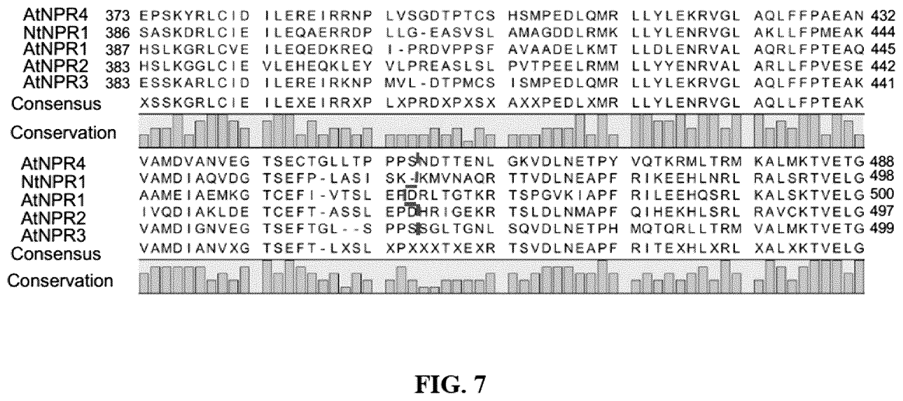

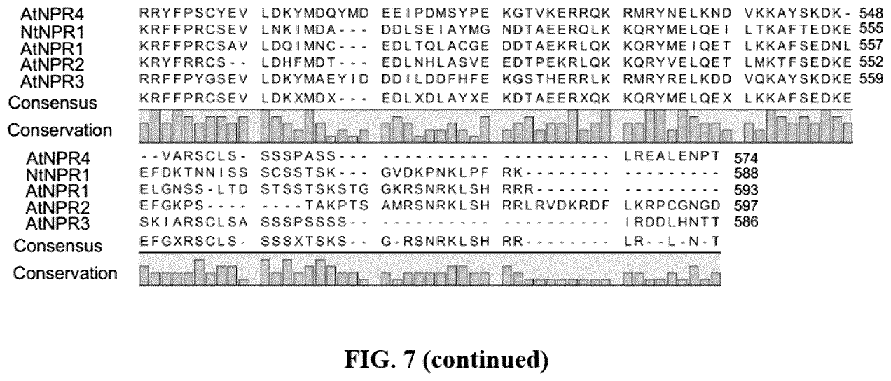

This idea was tested by splitting the SBC regions of AtNPR4 (amino acids 373-574) at a flexible loop region between S455 and N456 that connects α3 and α4 helices (FIG. 1; Panel b). Upon the addition of 1 mM SA, a moderate cytosol-to-PM translocation of mCh-AtNPR4-N was detected. Given that SA at mM concentrations tends to cause systemic toxicity, it became imperative for us to improve this CIP system by enhancing its SA binding strength. To achieve this, the assay was expanded to induce additional SBC domains from Arabidopsis NPR proteins (AtNPR1, AtNPR2, and AtNPR3) and tobacco NPR1 (NtNPR1). Guided by the primary sequence alignment results of these proteins, the native proteins were split at a loop position analogous to that in AtNPR4 (FIG. 7). Through a comparative analysis of all 25 combinations of NPR-N/C fragments derived from five plant NPR proteins, the pairing of NtNPR1-N and NtNPR1-C emerged as the most effective combination to drive PM translocation in the presence of 1 mM SA (FIG. 1; Panel b). This combination was thus selected for further refinement through three complementary strategies (FIG. 1; Panels c and d): (i) targeted mutagenesis at key residues surrounding the SA-binding site and at the interface mostly between the α2 and α4 helices; (ii) truncation of the N/C fragments to improve compactness; and (iii) random mutagenesis via error-prone PCR.

During the analysis, it was noted that co-expressing AtNPR3-N with AtNPR3-C failed to induce a response to SA. However, pairing AtNPR3-N with NtNPR1-C exhibited improved performance compared to the AtNPR4 N/C combination but was less effective than NtNPR1 N/C in the presence of 1 mM SA (FIG. 1; Panel b). Examination of the alignment results of their NPR-N domains revealed that the conserved penta-amino acid LENRV-like motif in AtNPR3 and AtNPR4 was LEKRV25, whereas the LENRV-like motifs of the other three NPR proteins featured asparagine at position 3. Given the crucial role of the LENRV-like motif in mediating SA sensing in NPR proteins, it was hypothesized that introducing the N430K mutation within the LENRV motif of NtNPR1 might enhance its SA binding strength. Indeed, cells expressing the N430K variant showed an increased response to SA compared to those expressing WT NtNPR1 (FIG. 1; Panel d). Encouraged by this finding, rational mutagenesis focused on the ligand-binding pocket and inter-helical interactions based on NtNPR1-N430K was conducted. The predicted structure of NtNPR1 (amino acids 386-588) revealed six closely packed α-helices, with the split site (K465/K466) dividing the first three N-terminal α-helices from the remaining three (FIG. 1; Panel c). Consequently, efforts were aimed at stabilizing the inter-helical interactions between these α-helices via site-directed mutagenesis to promote electrostatic and hydrophobic interactions. After testing 65 variants in HeLa cells, six key residues were identified (A400E, F482E, K485E, H488M, S496E, and D520E) that exhibited a larger response to 1 mM SA, resulting in an increase in PM translocation efficiency from 24% to nearly 50% compared to WT NtNPR1 (FIG. 1; Panel d). Combining these six mutations with N430K yielded the construct named NtNPR1-7M, which showed over a twofold increase in response to 1 mM SA (FIG. 1; Panel d). As cells expressing NtNPR1-7M demonstrated a nearly saturated response to 1 mM SA, the SA concentration was reduced to 10 μM for subsequent screening.

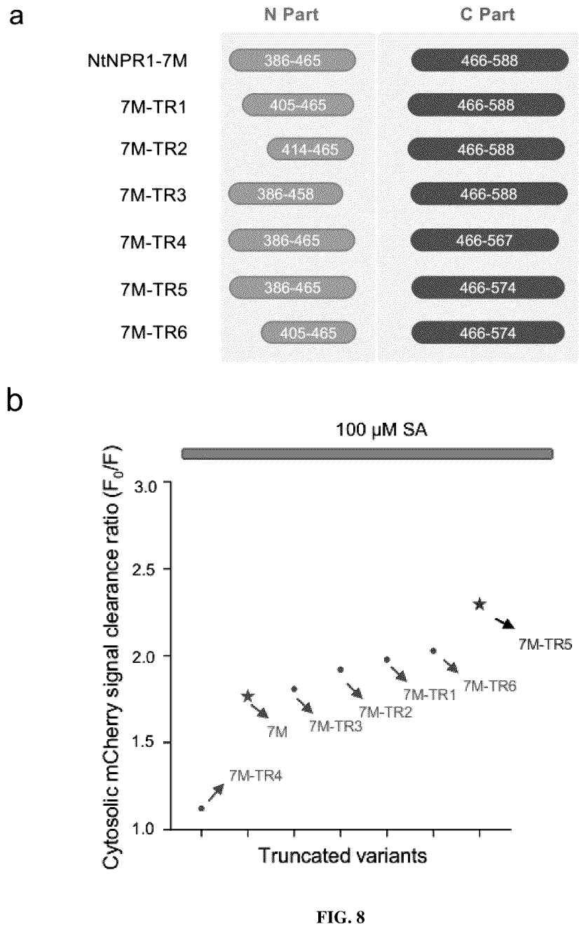

To delineate the minimal domains necessary for SA-induced heterodimerization, six combinations of truncated NtNPR1-7M N/C fragments were generated and their performance in HeLa cells were assessed (FIG. 8; Panels a and b). Among these constructs, NtNPR1-7M-TR5 emerged as the most promising candidate, displaying the highest degree of PM translocation upon exposure to 10 μM SA (FIG. 1; Panel d and FIG. 8; Panels a and b). Subsequently, randomized mutagenesis to further evolve NtNPR1-7M-TR5 was conducted. While most mutants displayed decreased SA responsiveness, two mutants, M423R and K430R, exhibited enhanced sensitivity to SA. Upon incorporating these two additional mutations into NtNPR1-7M-TR5 (thereafter designated as SAMBA), the most pronounced SA-induced PM recruitment was observed. By titrating increasing doses of SA into HeLa cells expressing SAMBA, the EC50 value of SAMBA for SA was established as 67.0±2.1 μM, which represents a remarkable potency enhancement of over 150-fold compared to the prototypical NtNPR1 (FIG. 1; Panel e). Notably, its EC50 falls within the lower spectrum of the therapeutic window of SA and aspirin after hydrolysis, yet remains far below the toxic threshold (>2 mM), suggesting its excellent biocompatibility and safety (FIG. 1; Panel f). Furthermore, the EC50 value surpasses by more than 30-fold the typical serum SA concentration detected in vegetarians (up to 1-2 μM), thereby mitigating the risk of SAMBA pre-activation when administered to mammals with a diet rich in SA-containing vegetables. This moderate affinity also renders SAMBA highly reversible, as evidenced by repeated PM translocation following multiple cycles of SA addition and subsequent washout. The activation and deactivation half-lives (t_{1/2}) of SAMBA were determined to be 57.4±3.1 s and 79.8±0.9 s, respectively (FIG. 1; Panel g).

Furthermore, appreciable PM translocation of SAMBA was also observed by incubating SAMBA-expressing HeLa cells with 10 μM aspirin (SA is the hydrolysis product of the FDA-approved OTC drug aspirin) for 2 hours at 37° C. (FIG. 1; Panel h). Interestingly, other SA derivatives with various modifications to the benzene group failed to exhibit robust responses at 10 μM (FIG. 1; Panel h), attesting to the specificity of SAMBA towards SA. Together, these data firmly establish SAMBA as a reversible SA-actuated heterodimerization system.

### Example 3—Reprogramming Protein Oscillations by SAMBA

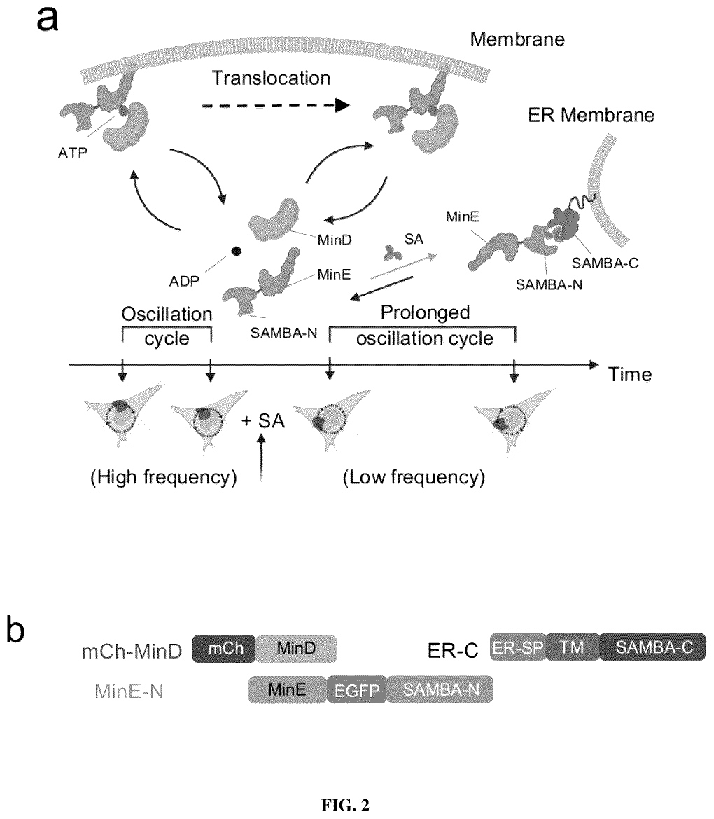

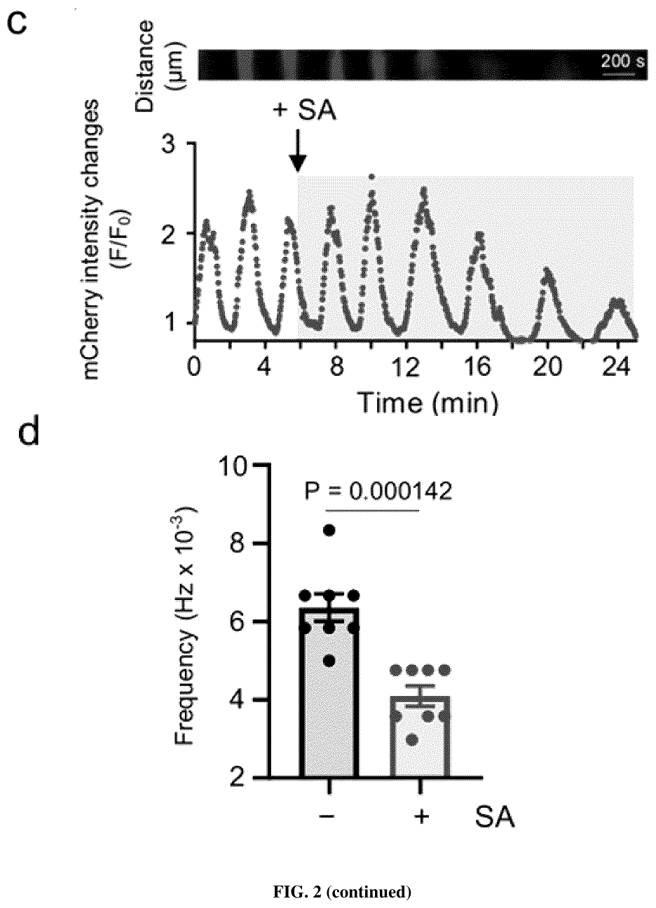

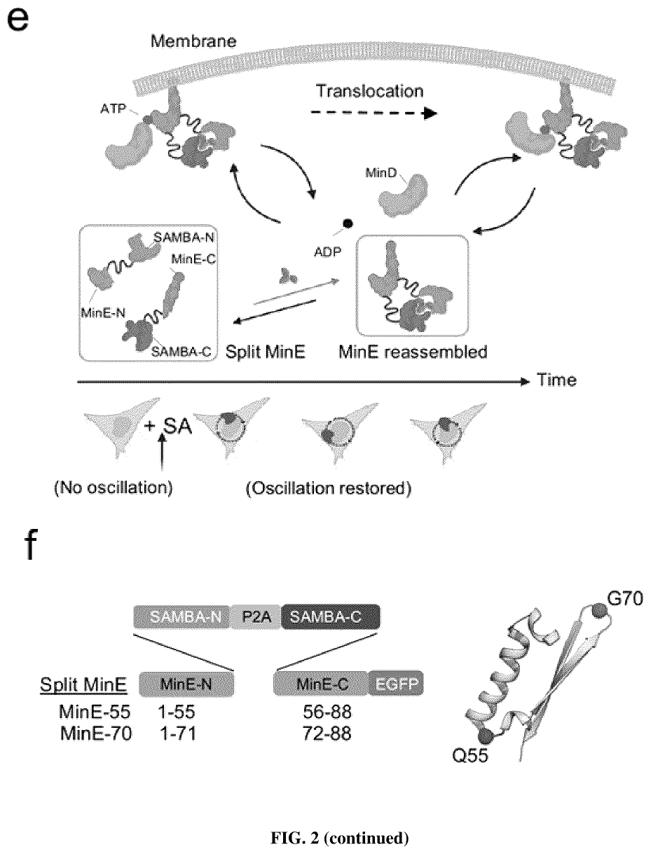

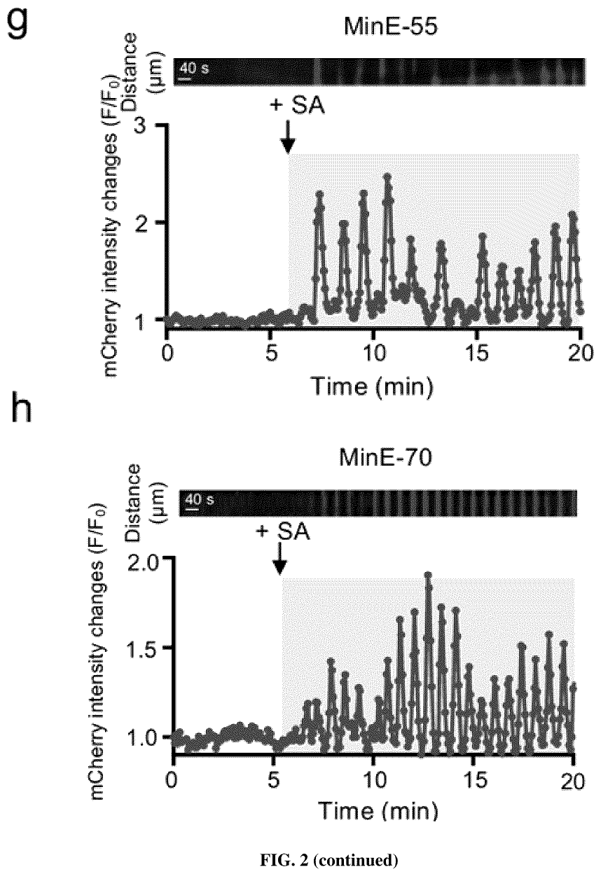

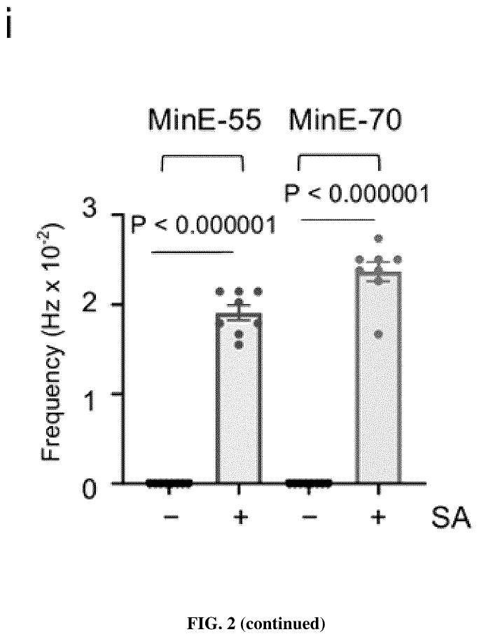

The genetically encoded bacterial MinD/MinE (MinDE) circuit provides an ideal system for exploring orchestration of protein dynamics within living cells. This two-component system comprises MinD, an ATPase, and MinE as an ATPase-activating protein (FIG. 2; Panel a). In rod-shaped bacteria, these proteins exhibit oscillatory behavior, moving from pole to pole and ultimately positioning the cell division scaffold protein, FtsZ, at the midcell of Escherichia coli. When MinD binds to ATP, it undergoes multimerization and attaches to the cell membrane, subsequently recruiting MinE to stimulate the ATPase activity of MinD. Following ATP hydrolysis, ADP-bound MinD freely diffuses into the cytoplasm, while MinE may either dissociate or transfer to another membrane-bound MinD. This MinDE system serves as a well-studied reaction-diffusion model in biology, extensively investigated in vivo, in vitro, and in silico. Ectopic expression of bacterial MinDE in mammalian cells has recently been shown to produce protein oscillations via repetitive binding to the cellular endomembrane, functioning as single-cell radios. Given that the MinD/MinE ratio regulates the frequency and amplitude of this reaction-diffusion system, it was contemplated that inducible tethering of one component toward a particular organelle, such as endoplasmic reticulum (ER), might alter the single-cell radio broadcasting frequency. To test this, MinE was fused with SAMBA-N and anchored SAMBA-C toward the cytosolic side of the ER membrane (FIG. 2; Panels a and b). Following SA addition, MinE molecules are anticipated to undergo cytosol-to-ER translocation to reduce the pool of free cytosolic MinE, consequently dampening the oscillatory frequency (FIG. 2; Panels a and b). Indeed, upon SA addition, pronounced recruitment of MinE toward the ER membrane was observed, accompanied by over 30% decrease in both the frequency and amplitude of minD oscillations (FIG. 2; Panels c and d).

Next, an alternative approach was explored to modulate MinDE oscillations by triggering the MinDE circuit reassembly in a ligand-dependent manner, which entails the direct engineering of SAMBA into the MinD or MinE proteins (FIG. 2; Panel e). Given the greater complexity and larger size of MinD compared to MinE, MinE was split and SAMBA was inserted into two exposed loop positions at Q55/I56 and G70/D71, with the resultant constructs termed MinE-55 and MinE-70, respectively (FIG. 2; Panel f). In the absence of SA, no appreciable MinDE oscillations were observed, with MinE evenly distributed throughout the cytoplasm. However, upon SA addition, the split MinE fragments reassembled into an intact functional protein due to the close proximity induced by SAMBA-N and SAMBA-C, subsequently initiating MinDE oscillations with an averaged frequency of 19 mHz for MinE-55 or 24 mHz for MinE-70 (FIG. 2; Panels g and h). Collectively, these results establish SAMBA as a robust CIP system capable of modulating protein oscillations either through SA-induced organellar tethering or the functional restoration of split proteins. These results also support SAMBA's application toward modulating other dynamic protein oscillation networks, including but not limited to control of actin dynamics, protein-based enzymes, and protein-protein interactions. For example, SAMBA has been inserted into a flexible loop region connecting the N- and C domains of SpvB, a microbial mono(ADP-ribosyl)transferase that antagonizes actin polymerization. In the absence of salicylic acid, the actin polymers were found to be intact due to the damaged function of split SpvB. However, the addition of salicylic acid induces the reassembly of the functional SpvB to restore its function—disrupting the actin cytoskeleton, and therefore killing cancer cells and mitigating neurodegeneration arising from aberrant actin modifications or perturbations.

### Example 4—Graded Gating of ORAI Ca²⁺ Channels by SAMBA

In this example, the ability of SAMBA to achieve tunable control over protein activities was evaluated. To test this effect, the calcium release-activated calcium (CRAC) channel was used, which consists of two components—the PM-embedded ORAI1 that forms the pore-forming subunit and the ER-resident stromal interaction molecule 1 (STIM1), which acts as the activator of ORAI1 Ca²⁺ channels. Forced juxtaposition of the N-terminus of the cytoplasmic domain of STIM1 (STIM1ct) has previously been shown to initiate conformational changes that overcome its intramolecular autoinhibition, thereby allowing STIM1ct to directly interact with and gate ORAI1 channels. The ensuing calcium flux further activates calcineurin (CaN), a calmodulin (CaM)-dependent phosphatase, which dephosphorylates NFAT to trigger its nuclear translocation and subsequent gene expression in T cells.

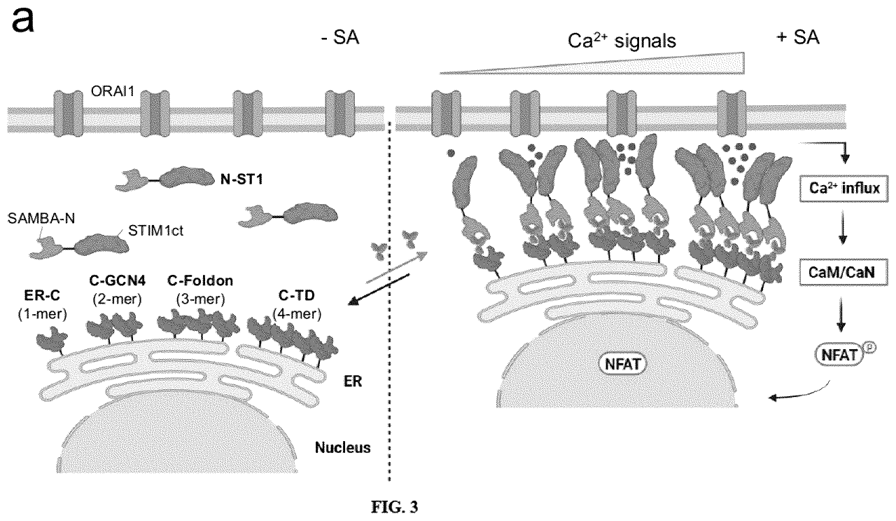

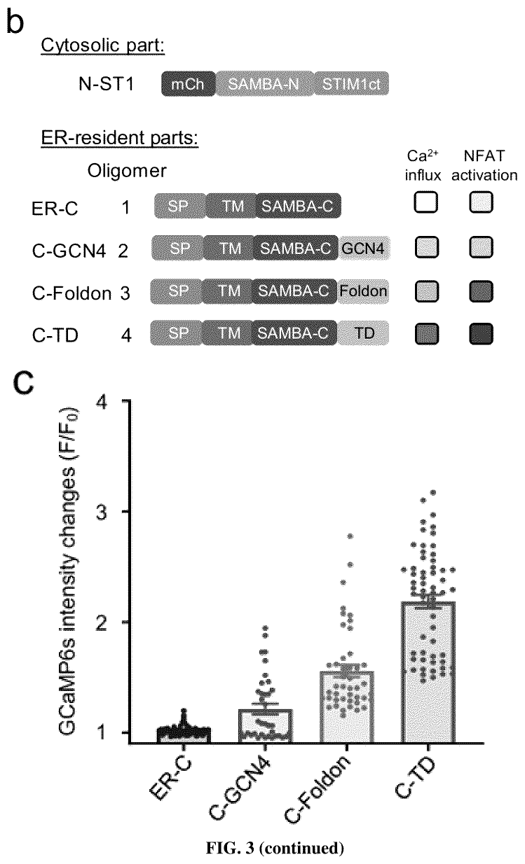

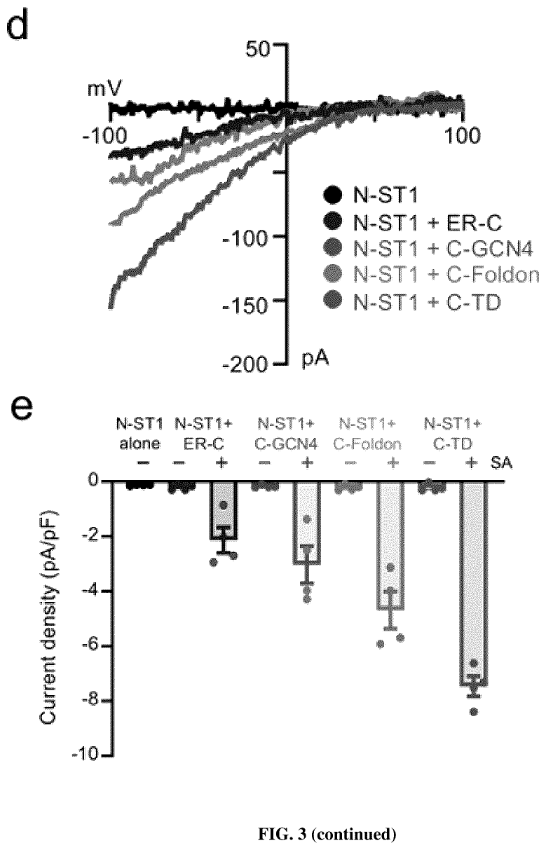

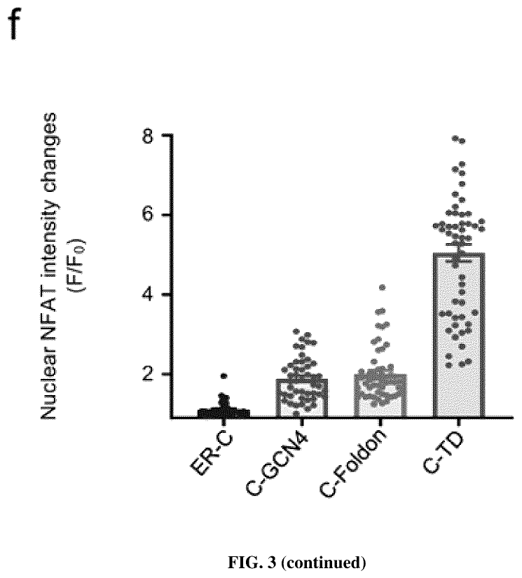

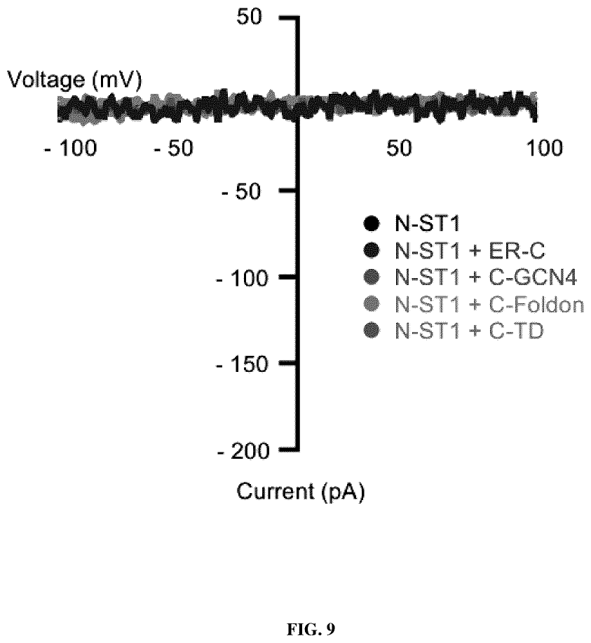

CRAC channel activation has been postulated to proceed through a graded process through the binding of different numbers of STIM1 molecules. It was reasoned that inducible oligomerization of STIM1ct at different oligomeric states towards the ER membrane would result in graded Ca2+influx and downstream signaling (FIG. 3; Panel a). To explore this, SAMBA-C was constrained in the ER membrane by fusing it with an ER signal peptide and the single transmembrane domain derived from STIM1. SAMBA-N was fused to STIM1ct (N-ST1) and remained in the cytosol in the absence of SA (FIG. 3; Panel b). To minimize potential influence on ER morphology and STIM1ct function due to large tags, the ER-resident SAMBA-C component was individually tagged with three small peptides: the 33-mer dimeric peptide derived from the leucine zipper domain of a yeast transcription factor General Control Nondepressible 4 (C-GCN4), a 27-mer trimeric foldon domain from bacteriophage T4 fibritin (C-Foldon), and a 36-mer tetramerization domain from human p53 (C-TD). Cells coexpressing ER-C and N-ST1 exhibited limited Ca²⁺ influx, despite evident SA-induced docking of N-ST1 towards the ER membrane. In contrast, cells coexpressing N-ST1 with the other three SAMBA-C variants showed an oligomeric state-dependent increase in Ca²⁺ influx, as evidenced by progressive rise in GCaMP6s signals when comparing among the C-GCN4, C-Foldon and C-TD groups (FIG. 3; Panel c). This trend was independently confirmed by electrophysiological measurements of CRAC channel currents (ICRAC). In CRAC-reconstituted HEK293 cells, a graded increase of ICRAC or current densities in cells expressing higher order SAMBA oligomers following SA treatment were observed (FIG. 3; Panels d and e). As control, cells expressing the same components without SA treatment showed negligible development of ICRAC (FIG. 9).

Given that Ca²⁺ influx through ORAI1 channels ultimately leads to the activation of downstream NFAT, the nuclear translocation of NFAT in a HeLa cell line stably expressing NFAT11-460-GFP was further evaluated. Upon comparing the degree of NFAT nuclear localization before and after SA treatment, a graded rise in nuclear localization of NFAT was observed following SA treatment (FIG. 3; Panel f), consistent with the trend seen in the Ca²⁺ influx assay and electrophysiological measurements. Overall, these results support the use of SAMBA oligomers in achieving tunable control over endogenous Ca²⁺ channels and the downstream signaling effector, which opens up new avenues for interrogating Ca²⁺-modulated physiological processes with high temporal precision and customizable signal outputs.

These results also support SAMBA's application toward modulating diverse ion channel complexes affording tunable control over cell signaling and gene expression. Examples of such ion channel complexes include but are not limited to SA-gated potassium channels, proton channels, sodium channels and chloride channels. For instance, SAMBA can be inserted into a viral K+ channel Kcv. When salicylic acid is added, the conformational changes of Kcv protein occur and further induce opening of the Kcv to induce changes in membrane potential to modulate the activity of excitable cells such as neurons, muscle cells, and cardiomyocytes.

### Example 5—SAMBA-Driven Activation of RTK Signaling and Gene Expression

After demonstrating the utility of SAMBA in modulating ion channels as described in Example 4, efforts were focused on engineering receptor tyrosine kinases (RTKs) to render them responsive to synthetic agonists like SA, thus obviating the necessity for natural cognate growth factors to trigger RTK activation.

Use of a caffeine-operated synthetic module (COSMO) to induce the dimerization of a PM-tethered intracellular kinase domain derived from RTK (RTK-ICD) with caffeine, thereby controlling the downstream signaling had been previously attempted. However, basal activation of RTK signaling even in the absence of caffeine was observed, consistent with findings from other studies on membrane-associated RTKs. This basal activity likely arises from the localization of myristoylated or palmitoylated RTK-ICD to lipid-rich microdomains, where high local concentrations of engineered receptors and other signaling components promote constitutive signaling activity in the absence of external stimuli. It was contemplated that the SAMBA platform could overcome the limitations of these other CIP systems. To test this, a SA-controllable RTK-ICD was engineered using the tropomyosin receptor kinase A (TrkA).

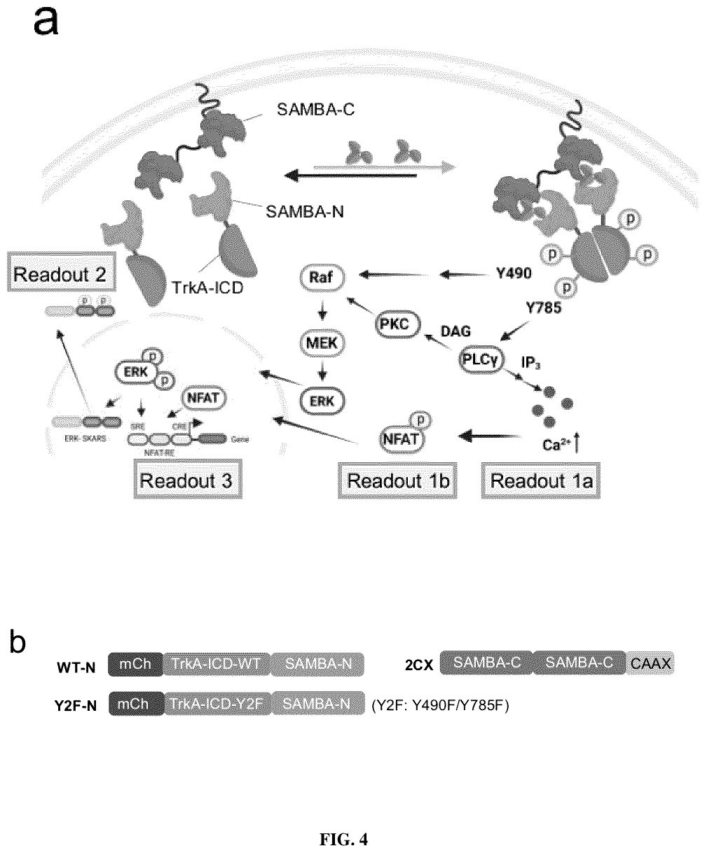

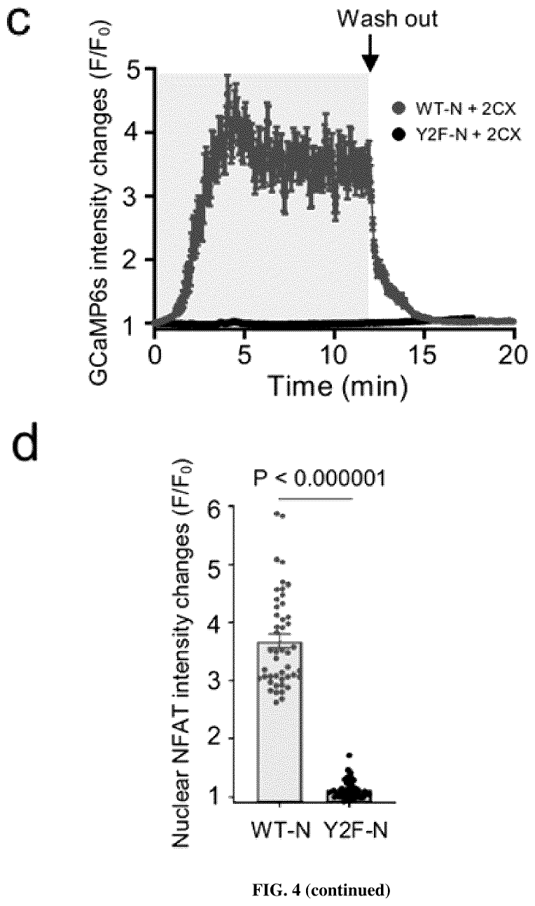

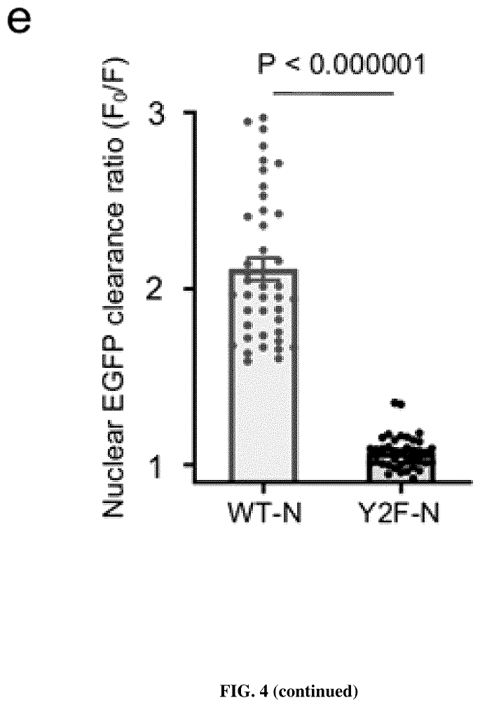

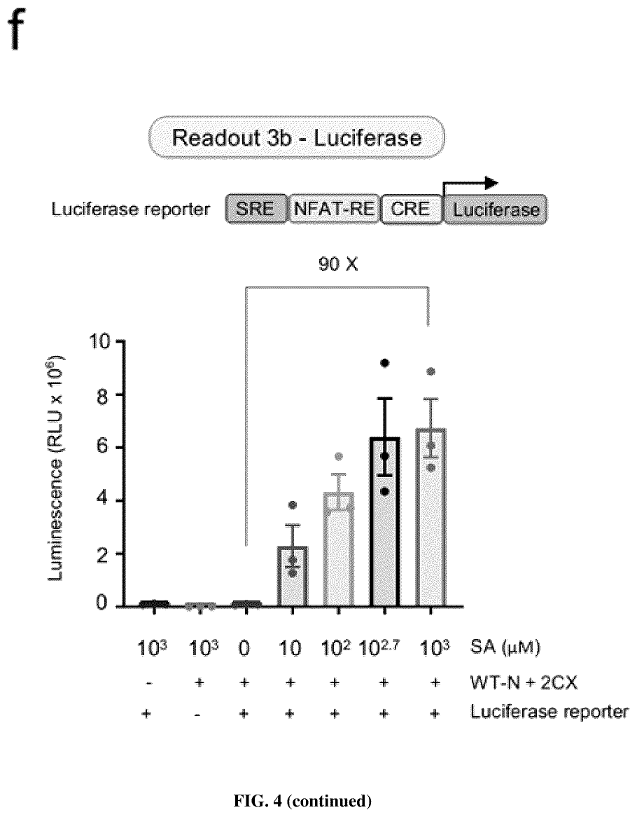

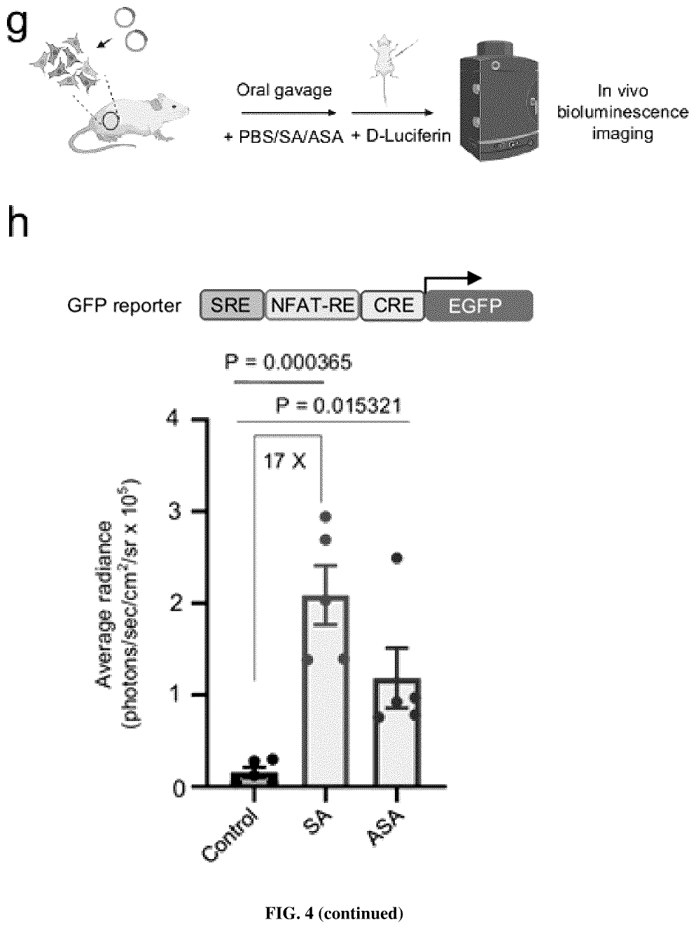

SAMBA-TrkA-ICD is designed to undergo SA-dependent translocation from the cytosol toward the inner half leaflet of the PM (FIG. 4; Panel a). The resting cytosolic distribution of SAMBA-TrkA-ICD, prior to SA treatment, is expected to minimize the basal activation of this engineered RTK. Conversely, SA-triggered subcellular translocation can induce the clustering of engineered TrkA-ICD near the PM, ultimately activating downstream effectors via the mitogen-activated protein kinase/extracellular-signal-regulated kinase (MAPK/ERK) and phospholipase C gamma (PLCγ) pathways (FIG. 4; Panel a). Two tandemly-connected SAMBA-C units were tethered to the PM using a CAAX motif (referred to as 2CX), while fusing wild-type TrkA-ICD with SAMBA-N within the cytoplasm (WT-N; FIG. 4; Panel b). A mutated version of TrkA-ICD containing the Y490F/Y785F substitutions (Y2F-N; FIG. 4; Panel b), designed to disrupt receptor multimerization-induced self-phosphorylation and subsequent ERK and PLCγ activation, served as a negative control. Upon SA addition, a robust increase in the cytosolic Ca²⁺ level was detected, as evidenced by a 4-fold elevation in GCaMP6s fluorescence (FIG. 4; Panel c), indicating inducible activation of the PLCγ pathway. In contrast, the inactive form of TrkA-ICD (Y490F/Y785F) did not exhibit SA-induced Ca²⁺ influx (FIG. 4; Panel c). Additionally, the influx of Ca²⁺ was reversed upon SA withdrawal, again illustrating the reversibility of SAMBA (FIG. 4; Panel c). Consistent with the Ca2+influx assay data, SA elicited efficient translocation of the downstream effector NFAT from the cytosol to the nuclei in cells expressing WT-N and 2CX, whereas no such translocation was noted in cells transfected with Y2F-N and 2CX (FIG. 4; Panel d). Concurrently, ERK activity was monitored using a synthetic kinase activity relocation sensor for ERK (ERK-SKARS), which reports ERK activation by shuttling from the nucleus to the cytosol upon ERK-mediated phosphorylation of nuclear localization sequences (NLS). A pronounced nuclear export of the ERK sensor in HeLa cells co-expressing WT-N and 2CX was noted, but not in those transfected with Y2F-N and 2CX (FIG. 4; Panel e).

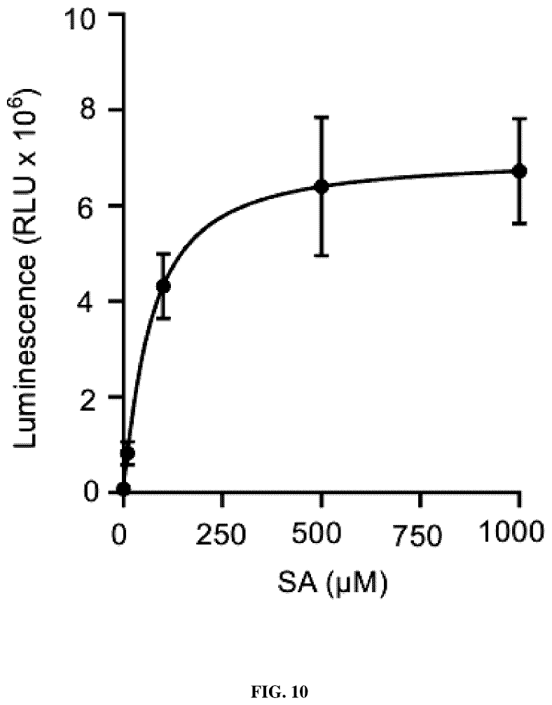

Having confirmed SA-inducible activation of both the PLCγ and ERK pathways without appreciable basal activity, the ability to harness the SAMBA-TrkA-ICD platform for tunable gene expression under the control of Ca2+/MAPK/ERK-response elements (RE), comprising serum response element (SRE), NFAT-RE, and cAMP response element (CRE), was explored. In HEK293T cells co-expressing WT-N, 2CX, and EGFP reporter, robust green fluorescence signals were detected after SA addition, while cells without SA treatment displayed minimal basal fluorescence. By using firefly luciferase-catalyzed bioluminescence as an alternate readout, a ˜90-fold increase of luminescence was observed in SA-treated cells compared to those without SA treatment (FIG. 4; Panel f), with the EC50 value determined to be 67.5±7.2 μM (FIG. 10). To further assess the in vivo compatibility of this system, engineered HEK293T cells co-expressing WT-N, 2CX, and luciferase were subcutaneously injected into the dorsal flanks of immunodeficient mice and treated the recipient mice with PBS (as control), SA, or aspirin (FIG. 4; Panel g). Compared to the control group showing negligible bioluminescence, both the SA and aspirin groups displayed strong bioluminescence signals. Considering that oral aspirin must undergo hydrolysis to convert into SA to exert its effect and that the effective serum SA concentration might be influenced by bioavailability, it is reasonable to expect that mice treated directly with SA would show more induction of luciferase activity compared to those receiving aspirin at a comparable dosage (FIG. 4; Panel h).

Collectively, this example shows that the SAMBA system can be successfully leveraged to recapitulate RTK signaling in a natural ligand-independent manner and achieve tunable transgene expression with minimal leakage, thereby establishing a robust approach for targeted gene expression in both in vitro and in vivo settings.

### Example 6—SAMBA-CAR Enables Programmable and Safer Immunotherapy

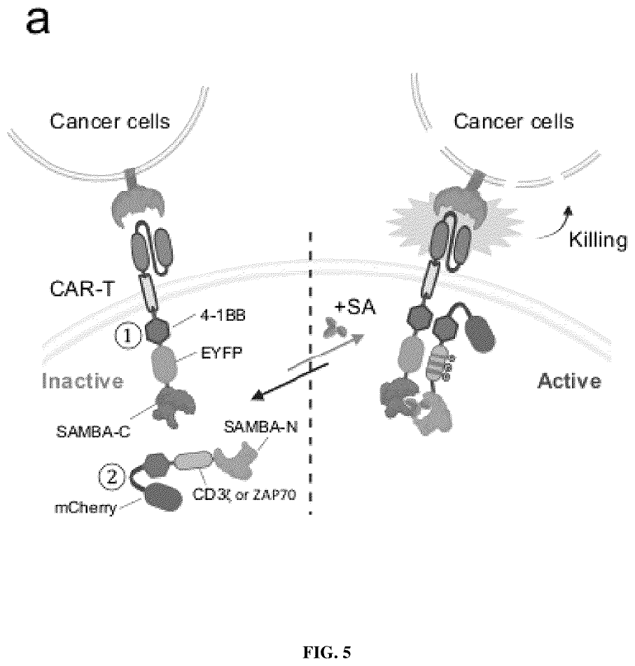

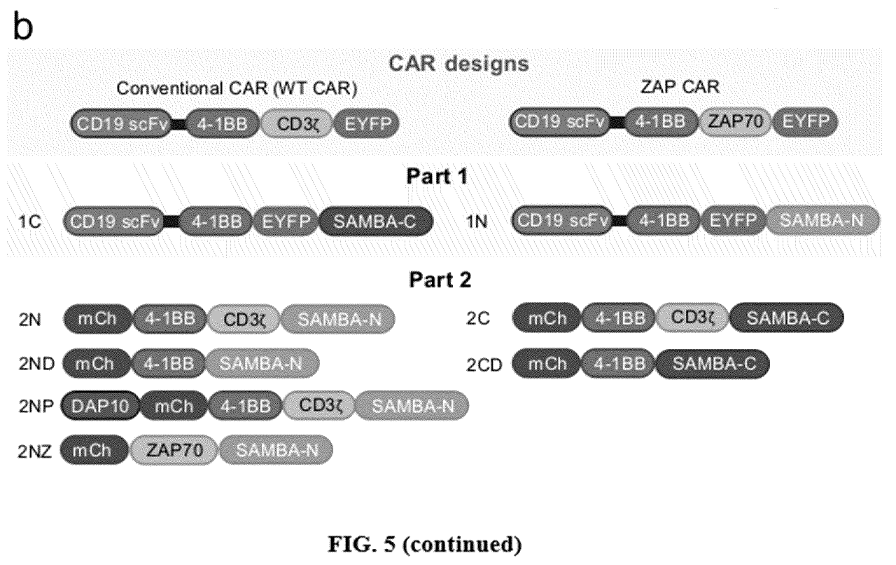

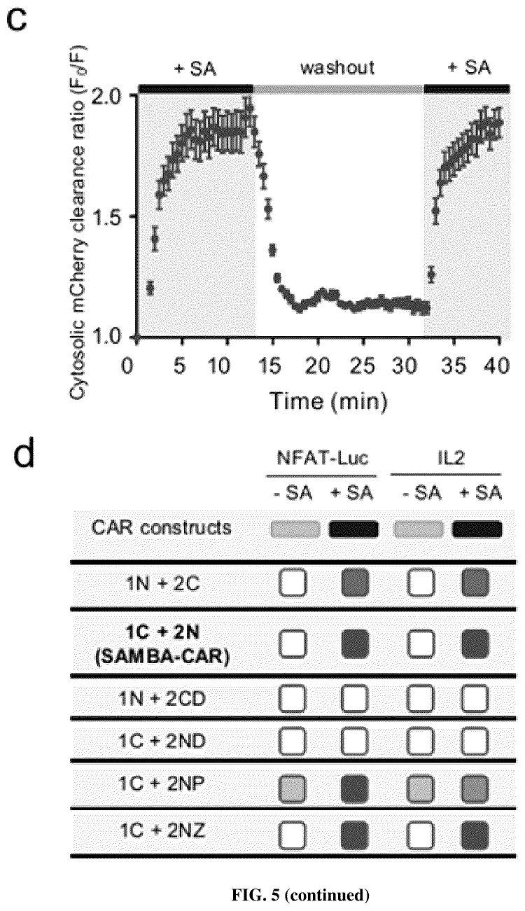

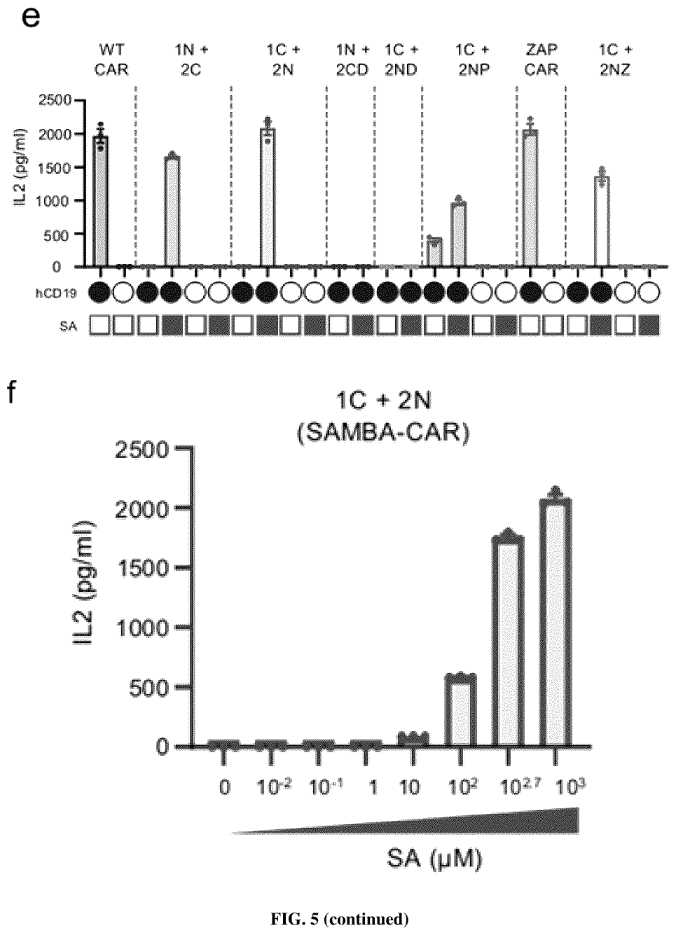

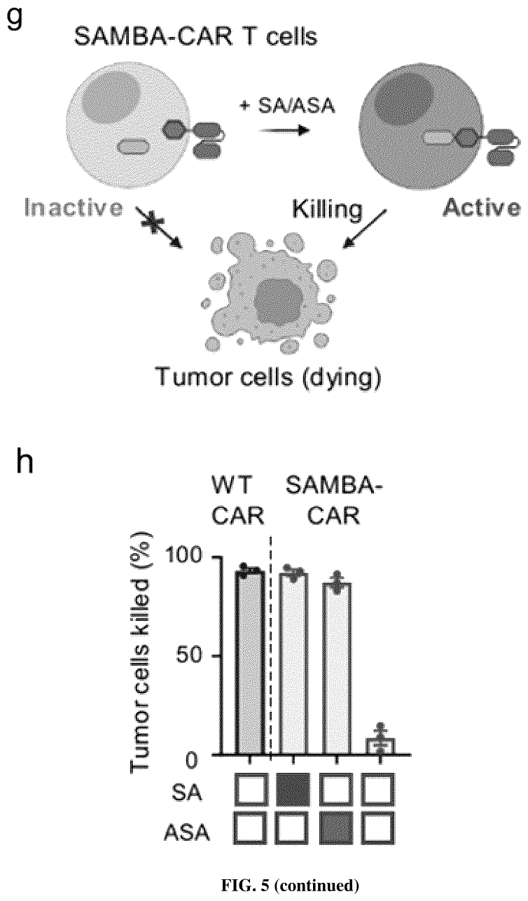

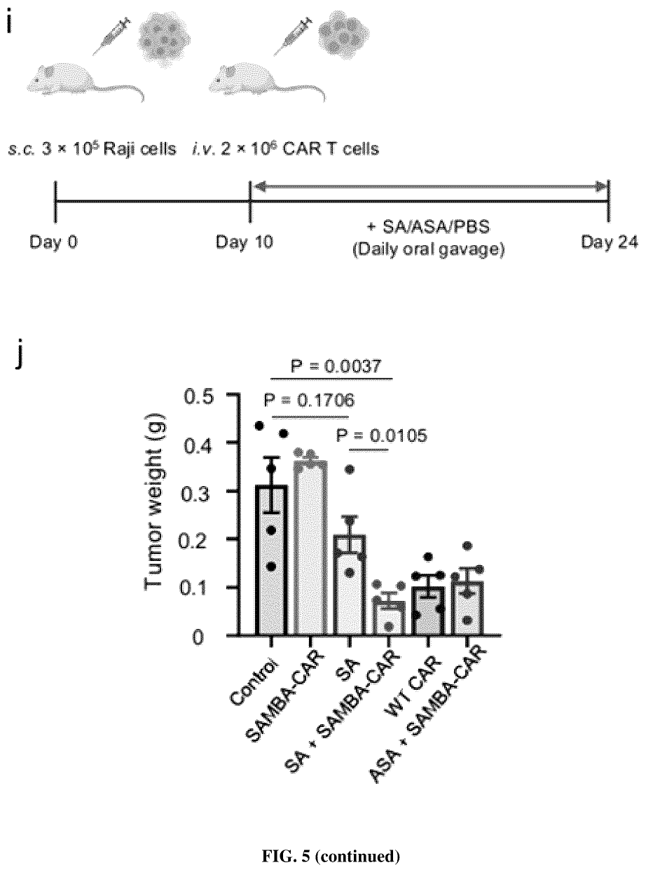

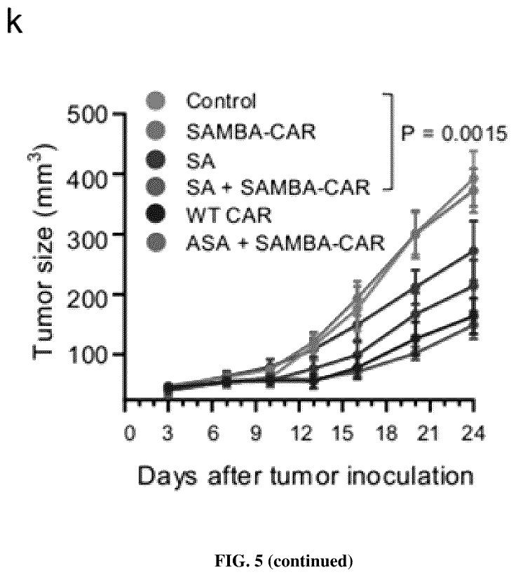

Based on the development and characterization of the SAMBA platform and its diverse applications, a SAMBA engineered chimeric antigen receptor (CAR) was further contemplated. A SAMBA CAR would enable SA-inducible T cell activation and precise tumor killing, while mitigating cytokine release syndrome (CRS) that is often associated with CAR T cell therapy. To test this, a series of hybrid constructs were designed by combining the N- or C-terminal fragments of SAMBA with the two split parts of an anti-CD19 CAR (referred to as Part 1 and Part 2; FIG. 5; Panels a and b). With the addition of SA, the reconstitution of a functional CAR near the PM was anticipated, thus allowing antigen engagement and subsequent T cell activation to kill tumor cells bearing the cognate antigen, such as CD19 (FIG. 5; Panel a). Part 1 was composed of an anti-CD19 single-chain variable fragment (scFv) in conjunction with GFP-tagged SAMBA-N or C components (constructs 1N/1C). Part 2 encompassed SAMBA-N or C components, and a 4-1BB costimulatory domain along with the T cell receptor-derived CD3ζ subunit, which facilitates T cell activation. Considering a recent development involving the use of ZAP70-CAR to bypass upstream signaling with less tonic signaling (Tousley, A. M. et al. Nature 615, 507-516 (2023)), Part 2 was also engineered by substituting CD3ζ with ZAP-70. To ascertain the optimal split CAR combinations, two variants of Part II were devised: one fused to a DAP10 transmembrane domain and integrated into the plasma membrane (construct 2NP), and the other remaining dispersed within the cytoplasm (constructs 2N/2C/2NZ; FIG. 5; Panel b). Furthermore, defective CAR constructs (2CD or 2ND) lacking either the T cell-activating CD3ζ subunit or the ZAP-70 component were incorporated to serve as stringent negative controls. SA-dependent reversible recruitment of cytosolic Part 2 toward PM-anchored Part 1 was first tested by co-expressing the two constructs in HeLa cells. As expected, prior to SA introduction, Part 2 (such as construct 2N) remained evenly distributed in the cytoplasm. Following SA introduction, a significant portion of the Part II protein underwent relocalization from the cytoplasm toward the PM, where the Part I protein was anchored. Moreover, the withdrawal of SA from the cell culture medium promptly reversed the cytosol-to-PM translocation of Part 2 within 2-3 minutes (FIG. 5; Panel c), further confirming the excellent reversibility of SAMBA.

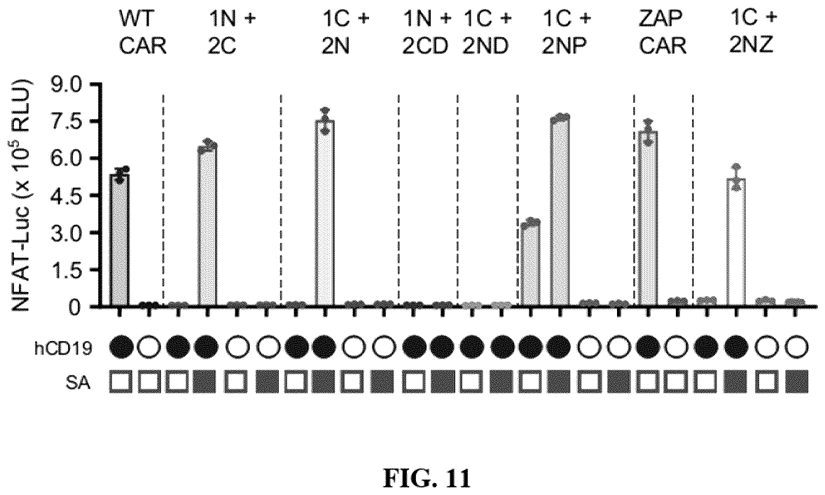

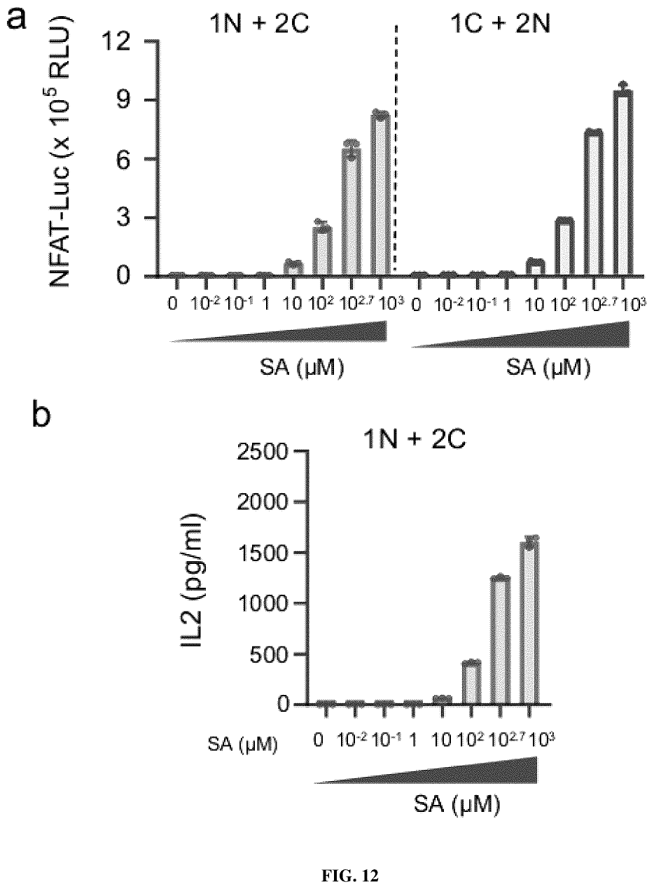

To screen the best combinations for enhancing the SA-inducible functional assembly of CARs in T cells, the two components were co-expressed in Jurkat cells stably expressing a luciferase reporter driven by the NFAT response elements (NFAT-Luc). Subsequent co-culture of engineered T cells with either human CD19 (hCD19)-negative K562 leukemia cells or hCD19+ Raji lymphoma cells allowed the measurement of luciferase expression and interleukin-2 (IL-2) production as independent indicators of T cell activation (FIG. 5; Panels d and f). When T cells expressing Part 1 on the PM and Part 2 in the cytoplasm (2N/2C/2NZ) were examined, minimal basal activation of engineered Jurkat cells was observed, as evidenced by negligible bioluminescent signals and IL-2 production in cells lacking SA or when exposed to hCD19-negative K562 cells (FIG. 5; Panels d and e, and FIG. 11). By contrast, pre-anchoring Part 2 to the PM (construct 2NP) resulted in pronounced pre-activation of Jurkat T cells in the absence of SA, thus this design was not pursued further in subsequent applications. Following SA addition, robust T cell activation was observed in the dual presence of SA and hCD19 antigen. Both the 1N+2C or 1C+2N combinations exhibited a dose-dependent response to SA in the co-culture assay (FIG. 5; Panel f, and FIG. 12; Panels a and b). Additionally, the 1C+2NZ combination successfully restored ZAP-70 CAR (ZAP CAR) T cell activation. The level of T cell activation seemed somewhat diminished in the 1N+2C (by 15.9%) or 1C+2NZ (by 30.7%) groups compared to their respective WT counterparts, whereas it appeared slightly elevated in the 1C+2N group (by 5.9%) (FIG. 5; Panel e). Since the 1C+2N combination (hereafter designated SAMBA-CAR) demonstrated superior performance compared to other groups, efforts were directed towards further characterizing SAMBA-CAR.

SAMBA-CAR was subsequently introduced into human T cells derived from peripheral blood mononuclear cells (PBMCs) to evaluate cytotoxicity against co-cultured CD19+ Raji tumor cells using SA or aspirin (FIG. 5; Panel g). As expected, SAMBA-CAR transduced T cells exhibited SA-dependent tumor cell killing, performing comparably to conventional CAR T cells. In the aspirin-treated SAMBA-CAR group, the efficiency of tumor killing was slightly reduced compared to the previous two groups (FIG. 5; Panel h). This might be attributed to the requirement for aspirin to be hydrolyzed into SA in order to exert its effect. To further assess the capability of SAMBA-CAR T cells for inducible tumor killing in vivo, a mixture of Raji lymphoma cells and Matrigel was subcutaneously implanted into the right flanks of SCID-beige mice. Following xenografts establishment at day 10, CAR T cells were administered to the mice through tail vein injection (i.v.). Over the following two weeks, mice were orally administered SA, ASA, or PBS on a daily basis (FIG. 5; Panel i). Notably, the SAMBA-CAR group treated with SA showed the most pronounced tumor-suppressive effects compared to the WT CAR group and the control groups that received blank T cells or SAMBA-CAR T cells without SA, evident in terms of both tumor weight and size (FIG. 5; Panel j and k). Interestingly, SA treatment alone also displayed a moderate suppression of tumor growth, consistent with existing literature documenting the tumor-suppressive effects of SA or aspirin across various cancers. Thus, SA seems to serve a dual role by triggering CAR-T cell activation and independently inhibiting tumor growth, thereby maximizing tumor killing efficacy. Furthermore, the SAMBA-CAR group treated with aspirin also exhibited reduced tumor burden, albeit less potent than SA (FIG. 5; Panels j and k). This disparity in efficacy may arise from suboptimal biodistribution and incomplete hydrolysis of aspirin into SA.

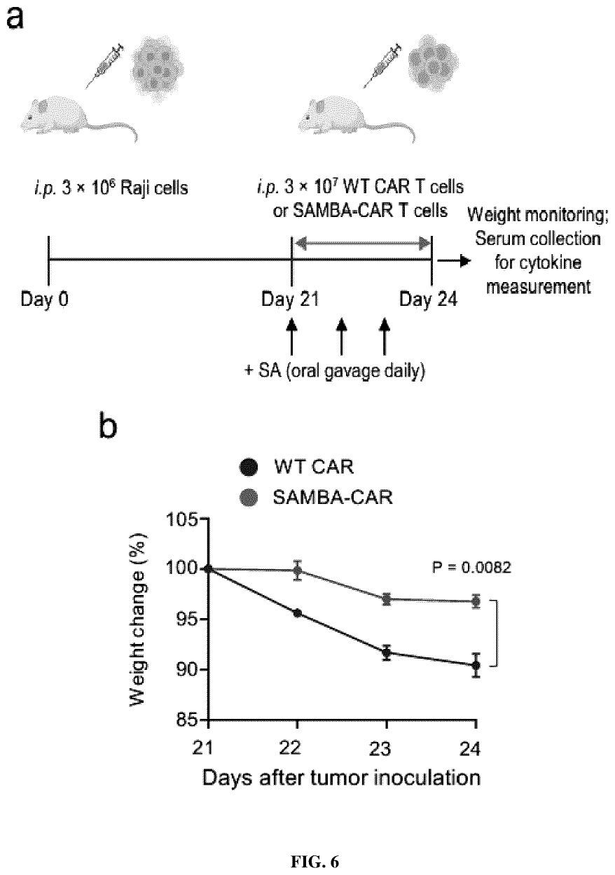

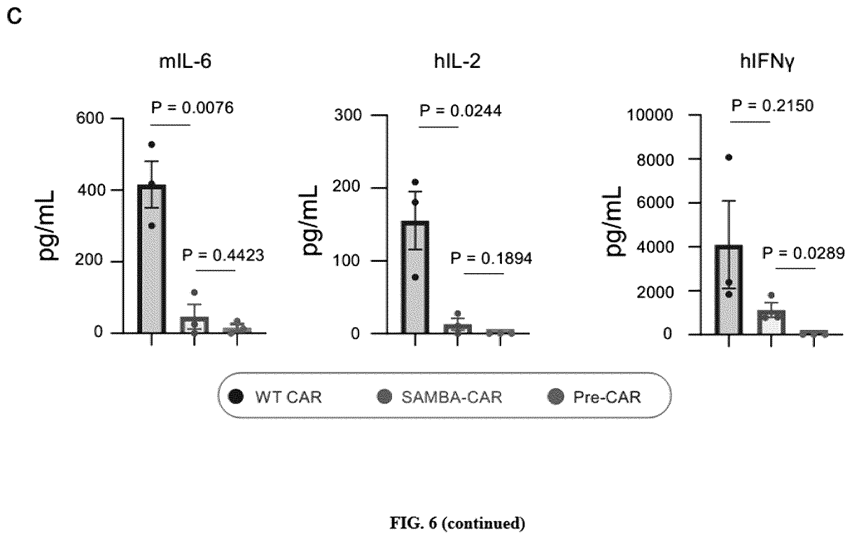

Given that cytokine release syndrome (CRS) emerges as the predominant acute toxicity following CAR-T cell therapy, SAMA-CAR T cells ability to effectively mitigate CRS was assessed by utilizing a well-established CRS mouse model (FIG. 6; Panel a) (Giavridis, T. et al. Nat. Med. 24, 731-738 (2018)). Three days after CAR-T cell infusion, mice receiving WT CAR T cells exhibited progressive weight loss. By contrast, those receiving SAMBA-CAR T cells and SA did not show significant weight loss (FIG. 6; Panel b). Furthermore, cytokine measurements in the sera of recipient mice revealed that the SAMBA-CAR group exhibited substantially lower release of CRS-associated cytokines, including mIL-6, hIFNγ, and hIL-2, when compared to the WT CAR group (FIG. 6; Panel c). These findings strongly indicate the potential of SAMBA-CAR in mitigating CRS in vivo. Congruently, the feasibility of utilizing SAMBA-CAR T cells for inducible lymphoma killing, both in vitro and in vivo, with reduced side effects.

### Example 7—SAMBA is a Widely Applicable Chemically Induced Proximity (CIP) System

The examples described herein demonstrate SAMBA to be a widely applicable chemically induced proximity (CIP) system that enables chemogenetic manipulation of diverse physiological processes in mammalian cells. The key strength of SAMBA lies in its ability to utilize SA or aspirin as the chemical switch to trigger reversible protein-protein heterodimerization. The minimal SAMBA variant, comprising only 189 amino acids, ranks among the most compact CIP systems and is compatible with various viral packaging platforms. Its small size also makes it easier for potential CRISPR-mediated integration into endogenous genomic loci. Additional applications of SAMBA will be tested in line with the proceeding Examples. For example, SAMBA will be operably linked to salicylic-controllable catalytically-inactive CRISPR-based base editing via inserting SAMBA into split adenine base editors (ABEs) like an evolved Escherichia coli tRNA adenosine deaminase (TadA). We anticipate that the addition of salicylic acid will restore the function of ABEs and show efficient and precise on-target single adenine editing and significantly reduced genomic and transcriptomic off-target effects. In addition, we can achieve SA-induced gene expression via inserting SAMBA between the catalytically inactive CRISPR/Cas9 (dCas9) and effector domains (such as VP64 or VPR). Once salicylic acid is added, the effector domain will be recruited to dCas9, which could further activate expression of therapeutic transgenes, such as chimeric antigen receptor for cancer cell targeting, cytokines (e.g., interleukin 2, interleukin 10, interleukin 12) and antibodies (e.g., anti-interleukin 6) for immunomodulation, insulin for diabetes intervention, genes, like insulin, atrial natriuretic peptide (ANP) for hypertension intervention, or huwentoxin-IV that acts as a safe and potent analgesic peptide to suppress the pain-triggering voltage-gated sodium channel NaV1.7.

The term “about” is used herein to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” When not used in conjunction closed wording in the claims or specifically noted otherwise, the words “a” and “an” denote “one or more.”

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has” or “includes” one or more steps is not limited to possessing only those one or more steps and also covers other unlisted steps. Similarly, any cell that “comprises,” “has” or “includes” one or more traits is not limited to possessing only those one or more traits and covers other unlisted traits.

While the disclosure has been described in connection with specific embodiments thereof, it will be understood that the present disclosure is capable of further modifications by one of skill in the art. It is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible. The present disclosure is therefore intended to encompass any variations, uses, or adaptations of the invention following, in general, the principles of the invention and including such departures from the present disclosure as come within known or customary practice within the art to which the invention pertains and as may be applied to the essential features herein before set forth.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

All publications, patents, and patent publications cited are incorporated by reference herein in their entirety for all purposes.

What is claimed is:

1. A method for inducing a protein-protein interaction, the method comprising:
a) obtaining a cell comprising a first and a second engineered salicylic acid receptor protein; and
b) exposing the cell to the presence of salicylic acid or a synthetic derivative to activate a protein-protein interaction between the first and a second engineered salicylic acid receptor protein in the presence of said salicylic acid or synthetic derivative thereof.

2. The method of claim 1, wherein the first engineered salicylic receptor protein comprises a polypeptide sequence having at least 85%, 90%, 95%, or 98% identity to a polypeptide selected from the group consisting of SEQ ID NOs: 6-18; and the second engineered salicylic receptor protein comprises a polypeptide sequence having at least 85%, 90%, 95%, or 98% identity to a polypeptide selected from the group consisting of SEQ ID NO:19-32.

3. The method of claim 1, wherein the first or second engineered salicylic receptor protein comprises:
a polypeptide sequence having an asparagine to lysine mutation corresponding to position 430 of SEQ ID NO: 1;
a polypeptide sequence having an alanine to glutamate mutation corresponding to position 400 of SEQ ID NO: 1;
a polypeptide sequence having an aspartate to glutamate mutation corresponding to position 520 of SEQ ID NO: 1;
a polypeptide sequence having a histidine to methionine mutation corresponding to position 488 of SEQ ID NO: 1;
a polypeptide sequence having a phenylalanine to glutamate mutation corresponding to position 482 of SEQ ID NO: 1;
a polypeptide sequence having a serine to glutamate mutation corresponding to position 496 of SEQ ID NO: 1;
a polypeptide sequence having a lysine to glutamate mutation corresponding to position 485 of SEQ ID NO: 1;
a polypeptide sequence having an asparagine to arginine mutation corresponding to position 430 of SEQ ID NO: 1;
a polypeptide sequence having an methionine to arginine mutation corresponding to position 423 of SEQ ID NO: 1; or
a combination of any thereof.

4. The method of claim 3, wherein:
the first engineered salicylic receptor protein comprises:
a polypeptide sequence having an asparagine to lysine mutation corresponding to position 430 of SEQ ID NO: 1;
a polypeptide sequence having an alanine to glutamate mutation corresponding to position 400 of SEQ ID NO: 1;
a polypeptide sequence having an asparagine to arginine mutation corresponding to position 430 of SEQ ID NO: 1;
a polypeptide sequence having an methionine to arginine mutation corresponding to position 423 of SEQ ID NO: 1; or
a combination of any thereof; or
the second engineered salicylic receptor protein comprises:
a polypeptide sequence having an aspartate to glutamate mutation corresponding to position 520 of SEQ ID NO: 1;
a polypeptide sequence having a histidine to methionine mutation corresponding to position 488 of SEQ ID NO: 1;
a polypeptide sequence having a phenylalanine to glutamate mutation corresponding to position 482 of SEQ ID NO: 1;
a polypeptide sequence having a serine to glutamate mutation corresponding to position 496 of SEQ ID NO: 1;
a polypeptide sequence having a lysine to glutamate mutation corresponding to position 485 of SEQ ID NO: 1; or
a combination of any thereof.

5. The method of claim 1, wherein the cell comprises a recombinant DNA construct encoding the first engineered salicylic acid receptor protein, the second engineered salicylic acid receptor protein, or the first and the second engineered salicylic acid receptor protein.

6. The method of claim 5, wherein:
the recombinant DNA construct is comprised within a vector;
the recombinant DNA construct is comprised within the cell's genome; or
the recombinant DNA construct encodes the first engineered salicylic acid receptor protein and a second recombinant DNA construct encodes the second engineered salicylic acid receptor protein.

7. The method of claim 1, wherein:
the first engineered salicylic acid receptor protein or the second engineered salicylic acid receptor protein is operably linked to a polypeptide sequence;
the protein-protein interaction between the first engineered salicylic receptor protein and the second engineered salicylic receptor protein modulates a cell signaling pathway;
the protein-protein interaction between the first engineered salicylic receptor protein and the second engineered salicylic receptor protein modulates T cell activation; or
the protein-protein interaction is fully reversible in the absence of said salicylic acid or the synthetic derivative thereof.

8. The method of claim 7, wherein the cell signaling pathway induces gene expression.

9. The method of claim 8, wherein the cell signaling pathway comprises an ion-dependent, a MAPK-dependent, an ERK-dependent, or a phospholipase C gamma-dependent signaling pathway.

10. A recombinant DNA construct encoding:
a) a first engineered salicylic acid receptor protein comprising a polypeptide sequence having at least 85%, 90%, 95%, or 98% identity to a polypeptide selected from the group consisting of SEQ ID NOs: 6-18;
b) a second engineered salicylic receptor protein comprising a polypeptide sequence having at least 85%, 90%, 95%, or 98% identity to a polypeptide selected from the group consisting of SEQ ID NOs: 19-32; or
c) the first engineered salicylic acid receptor protein and the second engineered salicylic acid receptor protein recited in a) and b).

11. The recombinant DNA construct of claim 10, wherein the recombinant DNA construct encoding the first engineered salicylic acid receptor protein or the second engineered salicylic acid receptor protein is operably linked in to a sequence encoding an enzyme, an antibody, a receptor, a transcription factor, a cytosolic protein, a membrane bound protein, or a proteasomal degradation component.

12. The recombinant DNA construct of claim 11, wherein:
the enzyme comprises a kinase;
the antibody comprises a single chain variable fragment;
the receptor comprises an engineered T cell receptor or co-stimulatory receptors; or
the proteasomal degradation component comprises an E3 ligase, SKP1, DDB1, SPOP, or TRIM21.

13. The recombinant DNA construct of claim 12, wherein:
the kinase comprises a receptor tyrosine kinases selected from the group consisting of TrkA, FGFR, TrkB, VEGFR, and EGFR;
the single chain variable fragment comprises a nanobody against mCherry, PD-1, PD-L1, Her-2, or CD19; or
the engineered T cell receptor or co-stimulatory receptors comprises an effector domain comprising a 41BB, a CD28, a OX40, a ICOS, or a CD3ζ chain.

14. The recombinant DNA construct of claim 10, wherein:
the recombinant DNA construct encodes two or more copies of the first engineered salicylic acid receptor protein, the second engineered salicylic acid receptor protein, or two or more copies of both the first and second engineered salicylic acid receptor proteins; or
the first engineered salicylic receptor protein and the second engineered salicylic receptor protein form a heterodimer in the presence of salicylic acid or a synthetic derivative thereof.

15. A cell comprising the recombinant DNA construct of claim 10.

16. The cell of claim 15, wherein the cell is a CAR T cell, a human cell, a bacterial cell, or a plant cell.

17. The recombinant DNA construct of claim 14, wherein the synthetic derivative is aspirin.

18. A method for activating a CAR T cell in a patient, the method comprising:
a) administering a CAR T cell to a patient, wherein the CAR T cell comprises a recombinant DNA construct encoding a first engineered salicylic acid receptor protein, a second engineered salicylic acid receptor protein, or a first and a second engineered salicylic acid receptor protein; and
b) exposing the cell to the presence of salicylic acid or a synthetic derivative to activate a protein-protein interaction between the first and a second engineered salicylic acid receptor protein in the presence of said salicylic acid or synthetic derivative thereof;
wherein activating the protein-protein interaction results in T cell activation.

19. The method of claim 18, wherein:
the recombinant DNA construct encoding the first engineered salicylic acid receptor protein or the second engineered salicylic acid receptor protein is operably linked in to sequence encoding a T cell receptor-derived subunit, a costimulatory domain, or a tyrosine kinase domain, or an E3 ligase complex component; or
the patient:
does not exhibit significant weight loss;
exhibits decreased release of CRS-associated cytokines; or
a combination thereof;
as compared to an appropriate control patient.

20. The method of claim 19, wherein:
the T cell receptor-derived subunit comprises a CD3ζ subunit;
the costimulatory domain comprises a 4-1BB domain, a CD28 domain, a OX40, or a ZAP70; or
the tyrosine kinase comprises the intracellular kinase domain of a tyrosine receptor kinase selected from the group consisting of TrkA, TrkB, EGFR, FGFR, VEGFR, and MET receptor tyrosine kinase.
